(19) United States
(12) Patent Application Publication
(10) Pub. No.: US 2026/0034223 A1
(43) Pub. Date: Feb. 5, 2026

(54) BIOMIMETIC EXTRACELLULAR MATRIX NANOFIBERS ELECTROSPUN WITH CALRETICULIN

(71) Applicants: NEW YORK University, NY, NY (US); The Trustees of the Stevens Institute of Technology, Hoboken, NJ (US)

(72) Inventors: Leslie I. Gold, NEW YORK, NY (US); Hongjun WANG, Millburn, NJ (US); Mary STACK, Bayonne, NJ (US)

(73) Assignees: NEW YORK University, New york, NY (US); The Trustees of the Stevens Institute of Technology, Hoboken, NJ (US)

(21) Appl. No.: 19/123,625

(22) PCT Filed: Oct. 24, 2023
(86) PCT No.: PCT/US2023/035821

§ 371 Date: Apr. 23, 2025

Related U.S. Application Data
(60) Provisional application No. 63/418,912, filed on Oct. 24, 2022.

(51) Int. Cl. A61K 47/42; A61K 31/727; A61K 31/728; A61K 38/39; A61K 47/18; A61K 47/34; A61K 47/36

(52) U.S. Cl. CPC A61K 47/42; A61K 31/727; A61K 31/728; A61K 38/39; A61K 47/18; A61K 47/34; A61K 47/36

(57) ABSTRACT

The present disclosure provides polymeric matrices comprising calreticulin (CRbT) and methods of producing and using such matrices. The polymeric matrices are useful in treatment of wounds (e.g., chronic diabetic wound).

### CROSS REFERENCE TO RELATED APPLICATION

This patent application claims priority to U.S. Provisional Application No. 63/418,912, filed on Oct. 24, 2022, the disclosure of which is incorporated by reference herein in its entirety.

### STATEMENT REGARDING FEDERALLY SPONSORED RESEARCH

This invention was made with government support under grant 32307-79 awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 12, 2023, is named 243735_000298_SL.xml and is 17,468 bytes in size.

### FIELD

The present application relates to polymeric matrices comprising calreticulin (CALR or CRT) and to methods of producing and using such matrices. The polymeric matrices are useful in treatment of wounds (e.g., chronic diabetic wound).

### BACKGROUND

Chronic wounds such as diabetic foot ulcers (DFUs) remain a devastating clinical challenge and cost the U.S. healthcare system over 25 billion dollars annually.¹ It has been estimated that 34% of people diagnosed with diabetes will develop a DFU over their lifetime.² DFUs resist most traditional healing treatments, showing a recurrence rate of 65% within 5 years after healing, an amputation rate of 15% within 1 year, and a five year mortality rate of 40%-50%. Clearly, DFUs present a substantial healthcare burden. Regranex, a gel containing platelet-derived growth factor-BB, a cytokine, is the only FDA-approved treatment for cutaneous wound repair. Whereas one study showed that 34% of DFUs treated with Regranex healed within 20 weeks,⁵ other studies in diabetic mice and treatment of DFUs showed lack of efficacy. Conversely, there are many cellular and tissue-based products (CTPs), formerly referred to as skin substitutes, on the U.S. market, but these also display less-than-desirable healing outcomes⁶⁻⁸. Most of the existing products are difficult to use and store, and have low cost-effectiveness. Accordingly, there is an unmet need to discover new therapeutic agents and methods of treatment that are useful for the healing of chronic wounds, including chronic diabetic wounds such as DFUs.

### SUMMARY

As specified in the Background section above, there is a great need in the art for therapeutic agents with enhanced wound healing properties. The present application addresses these and other needs.

In one aspect, provided herein is a polymeric matrix comprising calreticulin, or a functional fragment or derivative thereof.

In some embodiments of the polymeric matrix described herein, the derivative of calreticulin is a recombinant protein (e.g., human recombinant protein) comprising calreticulin or a functional fragment of calreticulin. In some embodiments, the functional fragment is N-, P- or C-domain of calreticulin.

In some embodiments of the polymeric matrix described herein, the polymeric matrix comprises a synthetic polymer, natural polymer, or a combination thereof. Examples of synthetic polymer include, but are not limited to, polycaprolactone (PCL), polylactic acid (PLA), poly(lactic-co-glycolic acid) (PLGA), poly(l-lactic acid)-co-poly(ε-caprolactone) (PLCL), polyphosphazene, poly-N-vinylpyrrolidone, polyglycolic acid, polydimethylsiloxane, poly(ethylene oxide)-poly(butylene terephthalate), nylon, polyvinyl alcohol (PVA), polyethylene glycol (PEG), or a combination thereof. Examples of natural polymer include, but are not limited to, collagen, chitosan, gelatin, hyaluronic acid, chondroitin sulfate, silk fibroin, elastin, tropoelastin, fibrin, fibrinogen, carboxymethyl cellulose, cellulose, decellularized tissue matrix, or a combination thereof.

In some embodiments, the polymeric matrix comprises PCL and collagen. The collagen may comprise type I collagen (Col1), type II collagen (Col2), type III collagen (Col3), type IV collagen (Col4), type V collagen (Col5), type VII collagen (Col7), or a combination thereof. In one embodiment, the collagen comprises type I collagen (Col1).

In some embodiments of the polymeric matrix described herein, the polymeric matrix comprises PCL and collagen at a weight-to-weight ratio of about 1:10 to 10:1. For example, the polymeric matrix may comprise PCL and collagen at a weight-to-weight ratio of about 1:10, 1:9, 1:8, 1:7, 1:6, 1:5, 1:4, 1:3, 1:2. 1:1, 2:1, 3:1, 4:1, 5:1, 6:1, 7:1, 8:1, 9:1, or 10:1. In one embodiment, the polymeric matrix comprises PCL and collagen at a weight-to-weight ratio of about 3:1.

In some embodiments of the polymeric matrix described herein, the polymeric matrix is in the form of nanofibers, foams, sponges, nonwoven meshes, spheres, hydrogels, or 3D printed filament structures. In one embodiment, the polymeric matrix is in the form of nanofibers.

In some embodiments of the polymeric matrix described herein, the nanofibers have a diameter of about 10-1000 nm. For example, the nanofibers may have a diameter of about 20-800 nm, about 50-750 nm, about 100-500 nm, about 200-400 nm, about 250-400 nm, about 300-400 nm, about 100 nm, about 200 nm, about 250 nm, about 280 nm, about 300 nm, about 310 nm, about 320 nm, about 330 nm, about 340 nm, about 350 nm, about 360 nm, about 370 nm, about 380 nm, about 390 nm, about 400 nm, about 500 nm, about 600 nm, about 700 nm, about 800 nm, or about 900 nm. In one embodiment, the nanofibers have a diameter of about 330 nm. In some embodiments, the nanofibers have a diameter of about 334 nm. In some embodiments, the nanofibers have a diameter of about 259-409 nm.

In some embodiments of the polymeric matrix described herein, the polymeric matrix comprises a concentration of calreticulin at about 1 pg-100 mg/mL. In some embodiments, the polymeric matrix comprises a concentration of calreticulin at about 1 pg-10 mg/mL, about 10 pg-1 mg/mL, about 100 pg-1 mg/mL, about Ing-1 mg/mL, about 10 pg-100 ng/ml, about 10 ng/ml, about 25 ng/ml about 50 ng/ml, about 75 ng/ml, about 100 ng/mL, about 150 ng/ml, about 200 ng/ml, about 250 ng/ml, about 300 ng/ml, about 400 ng/ml, or about 500 ng/ml. In some embodiments, the polymeric matrix comprises a concentration of calreticulin at about 100 ng/ml.

In some embodiments of the polymeric matrix described herein, the polymeric matrix further comprises an additional agent. In some embodiments, the polymeric matrix further comprises a cytokine, a growth factor, a glycosaminoglycan, a heat shock protein, a proteoglycan, a glycoprotein, syndecan, gelatin, or any mixtures thereof. In some embodiments, the glycosaminoglycan is hyaluronic acid. In some embodiments, the proteoglycan is perlecan or heparin sulfate. In some embodiments, the glycoprotein is fibronectin. In some embodiments, the growth factor is selected from the group consisting of a platelet-derived growth factor, vascular endothelial growth factor, fibroblast growth factor, epidermal growth factor, transforming growth factor-beta, and any mixtures thereof.

In some embodiments of the polymeric matrix described herein, the polymeric matrix has a three-dimensional structure.

In some embodiments of the polymeric matrix described herein, the polymeric matrix is produced by electrospinning.

In some embodiments of the polymeric matrix described herein, the polymeric matrix has one or more of the following characteristics:
1) induces proliferation and/or migration of keratinocytes;
2) induces proliferation and/or migration of fibroblasts;
3) induces expression of CD68 in monocytes;
4) induces TGF-β1, fibronectin, collagen, laminin-5, p-FAK, integrin α5 and/or integrin β1 and other extracellular matrix protein levels in fibroblasts and/or keratinocytes;
5) promotes a polarized cell shape, which is the morphological phenotype of a motogenic cell replete with lamellipodia and filopodia;
6) induces elongated and oriented cells on the CRT-NFs where CRT is presented at the basal side of the cell as well as the apical side;
7) induces dermal fibroblasts from the plantar foot of a non-healing wounds to adopt the phenotype of dermal fibroblasts from the plantar foot of a healing wound; and
8) induces tissue regeneration upon application to a mammal acute or chronic wound.

In one aspect, provided herein is a method of producing a polymeric matrix, comprising:
a) mixing calreticulin, or a functional fragment or derivative thereof, within a polymeric solution; and
b) fabricating the polymeric matrix from the solution generated in step (a) using electrospinning.

In some embodiments of the method described herein, the derivative of calreticulin is a recombinant protein (e.g., human recombinant protein) comprising calreticulin or a functional fragment of calreticulin. In some embodiments, the functional fragment is N-, P- or C-domain of calreticulin. In some embodiments, the calreticulin, or functional fragment or derivative thereof, is present in a solution comprising calreticulin, or functional fragment or derivative thereof, and a buffer.

In some embodiments of the method described herein, the buffer comprises an organic amine and a metal halide salt at a pH from about 6 to about 8. In some embodiments, the organic amine is tromethamine. In some embodiments, the metal halide salt is CaCl₂. In some embodiments, the buffer comprises saline, or PBS (phosphate buffered saline).

In some embodiments of the method described herein, the polymeric solution comprises a synthetic polymer, natural polymer, or a combination thereof. Examples of the synthetic polymer include, but are not limited to, polycaprolactone (PCL), polylactic acid (PLA), poly(lactic-co-glycolic acid) (PLGA), poly(l-lactic acid)-co-poly(ε-caprolactone) (PLCL), polyphosphazene, poly-N-vinylpyrrolidone, polyglycolic acid, polydimethylsiloxane, poly(ethylene oxide)-poly(butylene terephthalate), nylon, polyvinyl alcohol (PVA), polyethylene glycol (PEG), or a combination thereof. Examples of the synthetic polymer include, but are not limited to, natural polymer comprises collagen, chitosan, gelatin, hyaluronic acid, chondroitin sulfate, silk fibroin, elastin, tropoelastin, fibrin, fibrinogen, carboxymethyl cellulose, cellulose, decellularized tissue matrix, or a combination thereof.

In some embodiments of the method described herein, the polymeric solution comprises PCL and collagen. In some embodiments, the collagen can be any collagen, including but not limited to, comprises type I collagen (Col1), type II collagen (Col2), type III collagen (Col3), type IV collagen (Col4), type V collagen (Col5), or a combination thereof. In one embodiment, the collagen comprises type I collagen (Col1).

In some embodiments of the method described herein, the polymeric matrix comprises PCL and collagen at a weight-to-weight ratio of about 1:10 to 10:1. For example, the polymeric matrix may comprise PCL and collagen at a weight-to-weight ratio of about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the polymeric solution comprises PCL and collagen at a weight-to-weight ratio of about 3:1.

In some embodiments of the method described herein, the polymeric solution comprises a solvent. In some embodiments, the solvent is 1,1,1,3,3,3-hexafluoro-2-propanol (HFIP), trifluoroacetic acid, dichloromethane, or chloroform. In one embodiment, the solvent is 1,1,1,3,3,3-hexafluoro-2-propanol (HFIP).

In some embodiments of the method described herein, the mixing step (a) is carried out at a temperature of about 0 to about 25° C. In some embodiments, the mixing step (a) is carried out at a temperature of about 4 to about 10° C. In some embodiments, the mixing step (a) is carried out at a temperature of 0-20° C., 0-10° C., 4-10° C., 4-15° C., 4-20° C., 4-15° C., 1° C., 2° C., 3° C., 4° C., 5° C., 6° C., 7° C., 8° C., 9° C., or 10° C. In one embodiment, the mixing step (a) is carried out at a temperature of about 4° C.

In some embodiments of the method described herein, the final solution generated in step (a) comprises PCL and the solvent at a weight-to-volume ratio of about 1-20%. For example, the final solution generated in step (a) comprises PCL and the solvent at a weight-to-volume ratio of about 1-18%, 2-16%, 4-15%, 5-12%, 8-12%, or about 1%, 2%, 3%, 4%, 5%, 6%, 7%, 8%, 9%, 10%, 11%, 12%, 13%, 14%, 15%, 16%, 17%, 18%, 19% or 20%. In one embodiment, the final solution generated in step (a) comprises PCL and the solvent at a weight-to-volume ratio of about 10%.

In some embodiments of the method described herein, the fabrication step (b) is carried out at a temperature of about 20° C. to about 30° C. For example, the fabrication step (b) is carried out at a temperature of about 20° C., 21° C., 22° C., 23° C., 24° C., 25° C., 26° C., 27° C., 28° C., 29° C., or 30° C.

In some embodiments of the method described herein, the fabrication step (b) is carried out at a relative humidity of about 10% to about 60%. For example, the fabrication step (b) is carried out at a relative humidity of about 10%, 15%, 20%, 25%, 30%, 35%, 40%, 45%, 50%, 55%, or 60%.

In some embodiments of the method described herein, the electrospinning in step (b) is carried out using an electric field of about 0.5-20 kV/cm. For example, the electrospinning in step (b) is carried out using an electric field of about 0.5 kV/cm, 0.6 kV/cm, 0.7 kV/cm, 0.8 kV/cm, 0.9 kV/cm, 1 kV/cm, 1.5 kV/cm, 2 kV/cm, 2.5 kV/cm, 3 kV/cm, 3.5 kV/cm, 4 kV/cm, 4.5 kV/cm, 5 kV/cm, 5.5 kV/cm, 6 kV/cm, 6.5 kV/cm, 7 kV/cm, 7.5 kV/cm, 8 kV/cm, 8.5 kV/cm, 9 kV/cm, 9.5 kV/cm, or 10 kV/cm. In one embodiment, the electrospinning in step (b) is carried out using an electric field of about 1 kV/cm.

In some embodiments of the method described herein, the polymeric matrix is collected at a distance of 2 mm-50 cm. For example, the polymeric matrix is collected at a distance of 2 mm-50 cm, 5 mm-40 cm, 1 cm-30 cm, 2 cm-25 cm, 5 cm-20 cm, or 5 mm, 1 cm, 2 cm, 5 cm, 10 cm, 15 cm, 20 cm, 25 cm, 30 cm, 35 cm, 40 cm, 45 cm, or 50 cm.

In some embodiments of the method described herein, step (a) further comprises mixing an additional agent within the polymeric solution. In some embodiments, the additional agent is a cytokine, a growth factor, a glycosaminoglycan, a heat shock protein, a proteoglycan, a glycoprotein, syndecan, gelatin or any mixtures thereof. In some embodiments, the glycosaminoglycan is hyaluronic acid. In some embodiments, the proteoglycan is perlecan or heparin sulfate. In some embodiments, the glycoprotein is fibronectin. In some embodiments, the growth factor is selected from the group consisting of a platelet-derived growth factor, vascular endothelial growth factor, fibroblast growth factor, epidermal growth factor, transforming growth factor-beta, and any mixtures thereof.

In another aspect, provided herein is a polymeric matrix produced by the method described herein.

In another aspect, provided herein is a method for treating a wound in a subject in need thereof, said method comprises applying the polymeric matrix described herein to the wound in the subject. The polymeric matrix may be applied topically (e.g., on the surface of the wound) or internally to the wound (e.g., within the wound bed).

In some embodiments of the treatment method described herein, the wound is an acute wound or a chronic wound. In some embodiments, the acute wound is a burn, injury, or surgical intervention. In some embodiments, the chronic wound is a chronic diabetic wound, a venous or arterial stasis ulcer, a pressure ulcer, or an ulcer resulting from sickle cell disease (SCU). In some embodiments, the chronic diabetic wound is diabetic foot ulcer (DFU). In some embodiments, the wound is a post-surgical wound or an internal wound.

In another aspect, provided herein is a method for promoting healing of a chronic diabetic wound in a subject in need thereof, said method comprises applying the polymeric matrix described herein to the wound in the subject. In some embodiments, applying the polymeric matrix to the wound results in tissue regeneration in which neogenic epidermal appendage appear and scarring is reduced or eliminated. The diabetic wound may be diabetic foot ulcer (DFU). The polymeric matrix may be applied topically or internally to the wound.

In another aspect, provided herein is a method of inducing migration and/or proliferation of diabetic fibroblasts in a chronic diabetic wound in a subject in need thereof, said method comprises contacting the fibroblasts with the polymeric matrix described herein. The diabetic wound may be diabetic foot ulcer (DFU). The polymeric matrix may be applied topically or internally to the wound.

In some embodiments of the treatment method described herein, the method further comprises administering a cytokine, a chemokine, a growth factor, a glycosaminoglycan, a heat shock protein, a proteoglycan, a glycoprotein, gelatin, syndecan or any mixtures thereof. In some embodiments, the glycosaminoglycan is hyaluronic acid. In some embodiments, the proteoglycan is perlecan or heparin sulfate. In some embodiments, the glycoprotein is fibronectin. In some embodiments, the growth factor is selected from the group consisting of a platelet-derived growth factor, vascular endothelial growth factor, fibroblast growth factor, epidermal growth factor, transforming growth factor-beta, and any mixtures thereof.

In some embodiments of the treatment method described herein, the subject is mammal. In some embodiments, the subject is human.

### BRIEF DESCRIPTION OF THE DRAWINGS

FIGS. 1A-1C show incorporation of calreticulin (CALR or CRT) into polycaprolactone (PCL)/type 1 collagen (Col1) nanofibers (NFs). (FIG. 1A) Electron microscopy (SEM) images of PCL/Col1 NFs without CRT (PCX-0), PCL/Coll/CRT NFs containing 100 ng CRT (PCC-100n), and PCL/Coll/CRT NFs containing 1 μg CRT (PCC-1μ NFs). (FIG. 1B) Matching brightfield (left) and fluorescence (right) images of PCCf-100 NFs taken at the same field of view and magnification. Fluorescence is CRT-FITC. (FIG. 1C) ATR-FTIR spectra of NFs with or without CRT. Dashed vertical lines indicate the amide I band at 1665 cm⁻¹ and the amide II band at 1554 cm⁻¹. Semi-quantitation of the ratios of amide I to amide II: PCX-0=1.013. PCC-1n=1.034, PCC-10n=1.044, PCC-100n=1.172. FIG. 1D shows initial release experiment with PCCf-100n NFs on 15-mm diameter glass coverslips performed at 37° C. and with shaking at 80 rpm. Concentration was determined by measuring fluorescence of samples at indicated timepoints relative to a standard curve. PCX-0 NFs (gray squares) were included as a control. Three trendlines are fitted with R² indicated accordingly: third order polynomial of full dataset (dashed line), third order polynomial of truncated dataset starting at 4 h (dotted line starting at 4 h), linear of truncated dataset ending at 4 h (dotted line ending at 4 h). Bottom graph shows zoom in of 0.5-4 h timepoints.

FIGS. 2A-2C show sustained release of CRT from CRT-containing NFs. (FIG. 2A) Schematic illustration of experimental methodology used (FIG. 2B). Cumulative mass (in ng) of CRT-FITC released from PCCf-100n NFs on 15-mm diameter glass coverslips over time. Grayscale changes accompanied by black arrow heads indicate timepoints at which the receiver volume was fully replenished with fresh PBS. Insets: zoomed-in release graphs of first and final round. (FIG. 2C) Fluorescence images of PCCf-100n NFs and PCX-0 NFs (PCL-Col1 without CRT-FITC) submerged in PBS for different lengths of time (i.e., CRT-FITC remaining in PCL-Col1 following release into PBS over time until 8 weeks). Fluorescence above background of corresponding PCX-0 images is CRT-FITC. FIG. 2D shows CRT-FITC binds/adsorbs to nanofibers electrospun with 100 ng CRT (PCC-100n ) shown after 24 hours. CRT-FITC electrospun into NFs (PCCf-100) was shown to be released and the conditioned media containing CRT-FITC rebound or adsorbed to PCC-100n (CRT not fluoresceinated) at 37° C. without shaking. In the study shown in this Figure, CRT-FITC (90 μg/ml) was added to nanofibers containing 100 ng CRT (PCC-100n ) directly and after 24 hours, fluorescence imaging was performed using an EVOS microscope at 10× magnification.

FIGS. 3A-3C show proteolytic resistance of CRT-containing NFs. Effect of (FIG. 3A) elastase (at a molar ratio of 1:10) and (FIG. 3B) subtilisin (at a weight ratio of 1:10 and 1:100) on free CRT-FITC and PCCf-100n NFs submerged in PBS measured as fluorescence intensity of solution over time. (FIG. 3C) Fold change is presented as fluorescence after 275 min with enzyme relative to fluorescence of CRT-FITC [in solution] or PCCf-100n without enzyme.

FIGS. 3D and 3E show proteolytic susceptibility (more degradation of CRT-FITC) or resistance (less degradation of CRT-FITC) of FITC-CRT in the presence of cathepsin G and proteinase K. FITC-CRT, free in PBS solution (100 ng/0.7 ml) or incorporated into nanofibers (NFs) by electrospinning (PCC-100n; 15-mm diameter) and submerged in PBS were incubated with (FIG. 3D) cathepsin G (common enzyme in wound bed released by neutrophils and other cells) at a molar ratio of 1:10 (enzyme: CRT) or (FIG. 3E) proteinase K (broad substrate potent proteolytic enzyme) at a weight ratio 1:10 and 1:100 (enzyme: CRT). The reaction was carried out under static (without shaking) conditions at 37° C. Starting at 15 min and every 20 min thereafter until 4 h, a sample of each supernatant was collected followed by measuring its fluorescence intensity using a Biotek Synergy Neo2 Hybrid Multi-Mode Microplate Reader (excitation wavelength: 485 nm, emission wavelength: 528 nm). Not all data points are shown on the graph for better presentation.

FIGS. 4A-4C show proliferation of HFFs in response to various CRT conditions. (FIG. 4A) Phase contrast images of HFFs seeded at 5.2×10³ cells/cm² on NFs containing CRT (PCC-100n) or without CRT (PCX-0) and cultured for 7 d. (FIG. 4B) Quantified proliferation every 24 hours of HFFs seeded on NFs at 5.7×10³ cells/cm² and cultured over 3 days. NFs containing CRT (PCC-100n) were compared to NFs without CRT (PCX-0), with exogenous CRT added to cells on NFs that did not contain electrospun CRT (PCX-100n) and FBS (PCX-FBS) serving as controls. (FIG. 4C) Proliferation of HFFs in response to CRT released from NFs (conditioned media, CM) compared to unprocessed exogenously added CRT (exo), compared to control without CRT (no CRT). HFFs were seeded at 5.7×10³ cells/cm² on TCP (tissue culture plate/no NFs), then treated and cultured over 6 d. (FIG. 4B, FIG. 4C) * indicates p<0.05 and #indicates p<0.10 by means of unpaired t-tests assuming unequal variance compared to the control without CRT (PCX-0 in (FIG. 4B) or no CRT in (FIG. 4C)) at the same timepoint.

FIGS. 5A-5C show motogenic behavior of HFFs on CRT-containing NFs (CRT electrospun into PCL-Col1 NFs). The CRT-containing NF matrices are as described in Table 2 below. (FIG. 5A, FIG. 5B) Migration of HFFs on CRT-containing NFs using wound-gap closure assays. (FIG. 5A) Brightfield images of methylene-blue stained HFFs allowed to migrate on NFs for 51 h; initial gap is approximated by dashed vertical lines. (FIG. 5B) Quantified continuous gap closure of HFFs allowed to migrate on NFs over 91 h. Inset: 44 h timepoint; #indicates p<0.10 by means of unpaired t-test assuming unequal variances compared to the control without CRT (PCX-0) at the same timepoint. (FIG. 5C) Motility of HFFs on CRT-containing NFs by time-lapse photography using Bio-Tek Citation 10 Confocal Imaging Reader. Migration paths of 10 individual cells on NFs containing CRT (PCC-100n) or without CRT (PCX-0) over a minimum of 4 h; circles represent 320 μm and 640 μm distance marks.

FIGS. 6A-6F show protein expression of HFFs on CRT-containing NFs. (FIG. 6A, FIG. 6C) HFFs immunofluorescent stained for (FIG. 6A) pFAK, F-actin fibers, and cell nuclei or (FIG. 6C) vinculin, F-actin fibers, and cell nuclei after 24 h culture on NFs containing CRT (PCC-100n) or without CRT (PCX-0). White arrows indicate (FIG. 6A) pFAK+ vesicles (left panel) or (FIG. 6C) vinculin+ focal adhesions (left panel). Inset in (FIG. 6A): higher magnification of a single cell showing pFAK+ exosomes being secreted. (FIG. 6B) Quantification of the number of pFAK+ vesicles [outside of cells] found in (FIG. 6A) segregated by size. (FIGS. 6D-6F) Western blot analysis of HFFs after 48 h culture on CRT-containing NFs (PCC-100n) or corresponding controls (PCX-100n and PCX-0). Densitometric analysis of human foreskin fibroblasts seeded and grown on PCC-100n, PCX100n and PCX-0, prepared lysates electrophoresed by SDS-PAGE and immunoblotted with antibodies to the proteins shown (FIG. 6D) integrin β1, (FIG. 6E) fibronectin, and (FIG. 6F) TGF-β1 using β-actin as a control to ensure equal loading. Fold change was calculated over PCX-0. ** indicates statistical significance as p<0.01 and **** indicates p<0.0001 by means of unpaired t-tests compared to the control without CRT (PCX-0). FIG. 6G shows exosomes from HFFs on PCC-10n NFs as observed through high contrast brightfield image taken with Biotek Cytation C10. HFFs were seeded at 2.1×10² cells/cm² on the PCC-10n NF sample and cultured for 4 d.

FIGS. 7A-7C show proliferation of HEKs (human keratinocytyes) on CRT-containing NFs. (FIG. 7A) Quantified proliferation of HEKs seeded at 1.0×10⁴ cells/cm² on NFs and cultured over 5 d. NFs containing 100 pg CRT (PCC-100p) or NFs without CRT with 100 pg exogenous CRT (PCX-100p) were compared to NFs without CRT (PCX-0) serving as a control; * indicates p<0.05 by means of unpaired t-tests assuming unequal variances compared to the control without CRT (PCX-0) at the same timepoint. (FIG. 7B) HEKs immunofluorescently stained for Ki67 after 5-day culture on NFs containing CRT (PCC-100p) or without CRT (PCX-0). (FIG. 7C) HEKs immunofluorescently stained for F-actin fibers and cell nuclei after 8 d culture on NFs containing CRT (PCC-100p) or without CRT (PCX-0).

FIGS. 8A-8C show migration of HEKs on CRT-containing NFs. (FIG. 8A) Quantified continuous gap closure of HEKs allowed to migrate on NFs over 60 h in a wound-gap closure assay. (FIG. 8B) Zoom in on 60 h timepoint from (FIG. 8A); * indicates p<0.05 and #indicates p<0.10 by means of unpaired t-tests assuming unequal variances compared to the control without CRT (PCX-0) at the same timepoint. (FIG. 8C) HEKs immunofluorescently stained for laminin-5 (white arrows), F-actin fibers, and cell nuclei after 5-day culture from initial seeding density of 1.0 ×10⁴ cells/cm² on NFs containing CRT (PCC-100p) or without CRT (PCX-0).

FIGS. 9A and 9B show HFFs cultured on PCC-100n NFs induce integrin β1, fibronectin, and TGF-β1. Immunoblots shown here represent the graphs from FIG. 6D-F, which are densometric scanning of (FIG. 9A) integrin β1 and β-actin and (FIG. 9B) fibronectin, TGF-β1, and β-actin.

FIG. 10 shows the cell migratory response to exogenous CRT in the presence of Mitomycin C. HFFs, seeded at 2.3×10⁴ /well in 96 well plates, were incubated for 24 hours in complete MEM, switched to 0.5% FBS, fluorescently labeled with DiD according to manufacturer's instructions (Invitrogen V22887), and 700-800 μM, and wound gaps created using an Essen Bioscience Incucyte WoundMaker 96. The cells were treated with 5 μg/mL mitomycin C for 1 hour prior to being treated with 10 ng/ml CRT, untreated or treated with 5% FBS as the positive control. The cells were imaged intermittently over 5 days using an EVOS M7000 Imaging System. At the different time points, image J was used to crop images to around the initial gap and with adjusted contrast and variance filter. The raw images were used to create a binary image of the mask, which was quantitated as the per cent of gap closure determined as the change in the relative number of white pixels over time compared to the mask of 0 time point. Media was replaced after day 2. #=p<0.01 (one-sided t-test assuming equal variances); for CRT at −10 ng/ml treatment compared to no CRT at the same time points on the same day by single factor ANOVA. The data show that CRT mediated true migration as mitomycin C, which inhibits proliferation did not affect the CRT-induced migratory response. In contrast, the positive control, fetal bovine serum, shown on the graph as Exo-FBS, which contains many growth factors but classically used as a positive control in migration experiments, showed that migration was mainly due to proliferation.

FIG. 11A shows increased expression of alpha5 integrin by human foreskin fibroblasts (HFF) on calreticulin electrospun into nanofibers (PCC-100n); PCX-0 (PCL/Col1 NFs); PCX-100n (PCX with exogenous CRT added) over time (days 1, 2, and 4). HFFs were harvested from NF and cell lysates in RIPA buffer were immunoblotted for integrin alpha5. β1-actin is a loading control to ensure equal amounts of cell protein was added for each sample. Integrin alpha5 is a subunit integrin that combines with integrin beta 1, which is the fibronectin receptor for cellular migration on fibronectin substrate, as the provisional matrix for keratinocyte migration to resurface a wound.

FIG. 11B shows increased expression of beta-1 integrin over time by human foreskin fibroblasts (HFF) on calreticulin electrospun into nanofibers (PCC-100n); PCX-0 (PCL/Col1 NFs); PCX-100n (PCX with exogenous CRT added). HFFs were harvested from NFs and cell lysates in RIPA buffer were immunoblotted for integrin beta1. β-actin ensures equal loading. Integrin beta-1 is a subunit integrin that combines with other alpha integrins including alpha-5. Alpha-5beta-1 integrin is the fibronectin receptor for cellular migration on fibronectin substrate, as the provisional matrix for keratinocyte migration to resurface a wound. PCC-100n showed a greater induction of integrin betal by HFFs at an earlier time point (2 days) than PCX-100n (exogenous CRT) and PCX-0, NFs without CRT did not induce integrin betal. Thus, CRT is necessary for integrin betal induction on PCL/Col1 NFs.

FIG. 11C shows increased expression of TGF-β1 by human foreskin fibroblasts (HFF) on calreticulin electrospun into nanofibers (PCC-100n); PCX-0 (PCL/Col1 NFs); PCX-100n (PCX with exogenous CRT added). HFFs were harvested from NFs and cell lysates in RIPA buffer were immunoblotted with antibody to TGF-β1. β-actin ensures equal loading. TGF-62 1 induces ECM proteins such as fibronectin and collagens and is critical for the formation of granulation tissue for reconstructing the neodermis of the wound. PCC100n induced TGF-beta1 in HFFs at an earlier time point than that by exogenously added CRT to NFs (PCX-100n).

FIG. 11D shows dynamic collagen expression of HFFs on CRT-containing NFs. Western blot analysis of HFFs after 24, 48, 96 h culture on CRT-containing NFs. Densitometric analysis of immunoblots probed with anti-collagen antibodies. β-actin was used as a loading control and for normalizing the intensity of the band for collagen in each well. Fold change was calculated over respective PCX-0 at each time point. NFs without CRT did not induce collagen expression in HFFs. The data show steady production of collagen of HFFs grown on NFs containing CRT (PCC-100n) and NFs with exogenously added CRT (PCX-100n) or NFs alone (PCX-0).

FIGS. 12A and 12B show that calreticulin on NFs (PCC-100n) induced the surface expression of CD68 on macrophages treated with PMA (fluorescence) that were monocytes cultured with NFs containing CRT (PCC-100n). The expression of CD68 suggests that the monocytes have been activated into macrophages. Graph shows cell size in pixels2 and percent CD68 expression. PCX=NFs without CRT. Phorbol myristate acetate (PMA) induces macrophage activation and adhesion causing larger cell area.

FIG. 13 shows the structure activity relationship of calreticulin's wound healing activities. CRT can be expressed recombinantly as domains or fragments and retain certain wound healing activities that have been mapped to these individual domains. The domains/fragments show similar activities to intact protein in inducing the wound related activities shown by this diagram. Figure discloses SEQ ID NO: 10.

FIGS. 14A-14C show cultured normal (NFF14), healer (DFU7), and non-healer (DFU6) plantar foot fibroblasts cultured on nanofibers electrospun with CALR (CALR-NFS). FIG. 14A shows that at 6 days of culture, the normal (NFF14), healer (DFU7), and non-healer (DFU6) foot fibroblasts stained with phalloidin for visualization of actin fibers demonstrated a similar morphology grown on PCL/Col1 NF electrospun with CALR (CALR-NFs; PCC100n) (10× magnification). These cells (right panels) showed a regular elongated and aligned highly polarized morphology. The stained actin filaments appeared stretched across to the periphery of the cells; nuclei were stained with DAPI. This morphology typifies a motogenic phenotype compared to the plantar foot fibroblast cells cultured on PCL/Col1 NFs without CALR (NFs; PCX-0; left panels). In contrast, the cells grown on PCL/Col1 NFs without CALR, demonstrated an irregular stellate shape and were not elongated and aligned, as cells grown on CALR-NFs (PCC100n). FIG. 14B shows a higher magnification (40×) of the fibroblasts highlighting the notable elongated and aligned or oriented cells grown on CALR-NFs (PCC-100n; right panels) compared to the stellate morphology of cells grown on NEs without CALR (PCX-0; left panels). FIG. 14C shows, at 40× magnification at day 9 after seeding, the normal plantar foot fibroblasts (NFF14), DFU healer plantar foot fibroblasts (DFU7), and DFU non-healer plantar foot fibroblasts (DFU6) maintain an elongated, highly polarized, and aligned or oriented morphology on CALR-NFs (PCC-100n; right panels) compared to these same cells grown on PCL/Col1 NFs without CALR that show a more stellate morphology (left panels). Therefore, non-healer plantar foot fibroblasts show the same morphology of the healer dermal plantar foot fibroblasts showing the CRT-NFs (PCC100n) have instructed non-healer fibroblasts to adapt the motogenic/migratory phenotype of healer fibroblasts.

FIG. 15 shows quantified cell numbers of both non-healer (DFU6; black bars) and healer (DFU7; white bars) plantar foot fibroblasts. These data demonstrated an equal number of cells grown on NFs alone and CALR-NFs.

### DETAILED DESCRIPTION

Calreticulin (CALR or CRT) is an endoplasmic reticulum chaperone protein. The abbreviation CALR is interchangeable with CRT. The human calreticulin protein has been previously described and cloned, and has protein accession number NP_004334 (Fliegel, L. et al. (1989) J. Biol. Chem. 264:21522-21528; Baksh, S. et al., (1991) J. Biol. Chem. 266:21458-21465; Rokeach, L. A. et al., (1991) Prot. Engineering 4:981-987; Baksh, S. et al. (1992) Prot. Express. Purific. 3:322-331; Michalak, M. et al., (1992) Biochem. J. 285:681-692; Obeid M, et al (2007) Nature Medicine 13:54-61; Tesniere A et al. (2008) Curr Opin Immunol 20:1- 8; McCauliffe et al., J Clin Invest. 1990;86:332). Calreticulin has an amino terminal signal sequence, a carboxy-terminal KDEL ER retrieval sequence (SEQ ID NO: 10), multiple calcium-binding sites, and harbors three distinct domains N, P, and C within its 46,000 dalton molecular mass (401 amino acids) (Michalak, M. et al. (1999) Biochem. J. 344: Pt 2:281-292). Novel extracellular functions of calreticulin continue to be unraveled, portraying a protein with strong impact on developmental, physiological, and pathological processes (Bedard, K. et al. supra; Sezestakowska, D. et al. supra.; Michalak (2009) et al. supra). Calreticulin is localized to the surface of a variety of cells including platelets, fibroblasts, apoptotic cells, endothelial cells, and cancer cells and is required for the phagocytosis of apoptotic cells by all phagocytes (Gardai, S. J. et al (2005) Cell 123:321-334). Therefore, calreticulin functions in the removal of dead cells and tissue from wounds (debridement). The presence of dead tissue in a wound is a significant deterrent to the wound healing process. The presence of bacterial infection is also a critical deterrent to the healing of an acute wound injury or a chronic wound. Calreticulin enhances the uptake and ingestion of Staph. aureus by human neutrophils. This quality implicates a role for calreticulin as a bactericidal agent to fight infections in the wound bed. Calreticulin is also dynamically expressed during wound healing indicating its inherent importance in this process.

Topically applied CRT, as a protein, is challenging in terms of maintaining biological stability due to the deteriorated local environment of chronic wounds which introduce proteolytic^{13,14} and pH unstable¹⁵ conditions. Thus, repeated applications become essential in order to maintain the desirable functions, causing unwanted inconvenience in clinical practice and unnecessary consumption of more CRT. In this regard, it would be highly beneficial to have CRT sustainably released locally while protecting it from environment challenges.

In some aspects of the present disclosure, polycaprolactone (PCL)/type I collagen (Col1) electrospun fibers were investigated for use as an effective carrier for calreticulin (CRT), particularly in regard to retaining its biological activity, protecting it from proteolytic degradation, and sustaining its release, while at the same time introducing further wound healing capability through the synergy of biological and physicochemical cues. Results reported herein showed that CRT could be uniformly incorporated into PCL/Col1 fibers by direct blending into the electrospinning solution, forming PCL/Col1/CRT (PCC) nanofibers (NFs). Importantly, this blending process neither altered the diameter distribution of obtained NFs nor caused the bioactivity loss of CRT. CRT was able to sustainably elute from these NFs at physiological conditions (37° C. in pH 7.4 PBS) and was persistently present within NFs as long as 8 weeks. Upon challenge by enzymatic exposure, CRT in NFs exhibited greater resistance against proteolytic degradation. While there was no statistically significant improvement in a gap closure assay with fibroblasts, PCC NFs facilitated faster and more complete gap closure with keratinocytes. The untreated control HFFs showed significant random migration compared to CRT, which showed CRT directed migration (standard deviations were small compared to the untreated control). Furthermore, PCC NFs elicited a motile cell phenotype as seen through cell polarization and laminin-5 deposition by keratinocytes and cell polarization, vinculin capping F-actin fibers, and phosphorylated focal adhesion kinase (pFAK) localization in fibroblasts. PCC NFs also promoted proliferation of both fibroblasts and keratinocytes. In addition, PCC NFs also upregulated the synthesis of several wound-healing related protein markers (integrin-β1, fibronectin, and transforming growth factor (TGF)-β1). Taken together, the PCC hybrid NFs have potential for healing recalcitrant wounds such as DFUs with a synergistic action that is not observed with PCL/Col1 NFs or with CRT alone. These findings are exhibited in Stack, M. E., et al. ACS Appl. Mater. Interfaces, 2022, 14, 46, 51683-51696, the contents of which are incorporated herein by reference in their entireties.

### Definitions

“Treat” or “treatment” as used herein in connection with wound healing means improving the rate of wound healing or completely healing a wound. Methods for measuring the rate of wound healing are known in the art and include, for example, observing increased rate of epithelialization with the formation of all 4 layers of the epidermis and/or granulation tissue formation, or lessening of the wound diameter and/or depth. Increased epithelialization can be measured by methods known in the art such as by, for example, the appearance of new epithelium at the wound edges and/or new epithelial islands migrating upward from hair follicles and sweat glands (as shown in the healing of human and porcine wounds). Granulation tissue is necessary for proper healing and for providing a scaffold for the migration of keratinocytes over the wound for resurfacing and for tissue remodeling including filling in the wound defect. The amount of area of granulation tissue formation can be measured by morphometric analysis by measuring the area of the granulation tissue, which can be referred to as neodermis.

“Chronic wound” as used herein means a wound that has not completely closed in eight weeks since the occurrence of the wound in a patient having a condition, disease or therapy associated with defective healing. Conditions, diseases or therapies associated with defective healing include, for example, diabetes, arterial insufficiency, venous insufficiency, chronic steroid use, cancer chemotherapy, radiotherapy, radiation exposure, and malnutrition. A chronic wound includes defects resulting in inflammatory excess (e.g., excessive production of Interleukin-6 (IL-6), tumor necrosis factor-alpha (TNF-α), and MMPs), a deficiency of important growth factors needed for proper healing, bacterial overgrowth and senescence of fibroblasts. A chronic wound has an epithelial layer that fails to cover the entire surface of the wound and is subject to bacterial colonization, which can result in biofilm formation, which is resistant to treatment with anti-bacterial agents.

“Chronic diabetic wound” means a chronic wound in a patient with diabetes. A chronic diabetic wound may be associated with peripheral neuropathy and/or macro-and micro-vascular insufficiency. A diabetic foot ulcer is one type of chronic diabetic wound.

The term “hyaluronic acid” (HA) as used in the present application refers to hyaluronic acid or salts of hyaluronic acid, such as the sodium, potassium, magnesium and calcium salts, among others. The term “hyaluronic acid” is also intended to include not only elemental hyaluronic acid, but hyaluronic acid with other trace of elements or in various compositions with other elements, as long as the chemical and physical properties of hyaluronic acid remain unchanged. In addition, the term “hyaluronic acid” as used in the present application is intended to include natural formulas, synthetic formulas or combination of these natural and synthetic formulas. Non-limiting examples of useful hyaluronic acid preparations which can be used in the methods of the present invention include, for example, Juvederm® (a highly-crosslinked hyaluronic acid product sold by Allergan, Inc.).

“Patient” or “subject” refers to mammals and includes human and veterinary subjects.

A “therapeutically effective amount” means the amount of a compound that, when administered to a mammal for treating a chronic diabetic wound, is sufficient to effect such treatment. The “therapeutically effective amount” may vary depending on the size of the wound, and the age, weight, physical condition and responsiveness of the mammal to be treated.

As used herein, the term “promote wound healing” is used to describe an agent that increases the rate at which a wound heals and the quality of wound repair.

The term “growth factor” can be a naturally occurring, endogenous or exogenous protein, or recombinant protein, capable of stimulating cellular proliferation and/or cellular differentiation and cellular migration.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

The term “calreticulin”, “CRT”, and “CALR” are used interchangeably herein.

“Electrospinning,” as used herein, refers to a process wherein a high voltage electric field is generated between oppositely charged polymer fluid contained in a glass syringe with a capillary tip and a metallic collection screen. As the voltage is increased, the charged polymer solution is attracted to the screen. Once the voltage reaches a critical value, the charge overcomes the surface tension of the suspended polymer cone formed on the capillary tip of the syringe and a jet of ultra-fine fibers is produced. As the charged fibers are sprayed, the solvent quickly evaporates, and the fibers are accumulated randomly on the surface of the collection screen. This results in a nonwoven mesh of nano and micron scale fibers. Varying the charge density (applied voltage), polymer solution concentration, solvent used, and the duration of electrospinning can control the fiber diameter and mesh thickness. Other electrospinning parameters which may be varied routinely to affect the fiber matrix properties include distance between the needle and collection plate, the angle of syringe with respect to the collection plate, and the applied voltage. Micro and nanofibers with wide ranges of diameters from 1-999 nm to within the micron range can be obtained by varying various experimental parameters such as viscosity of the polymer solution, electric potential at the capillary tip, diameter of the capillary tip as well as the gap or distance between the tip and the collecting screen.

“Nano-” as used herein, generally refers to structures having dimensions that may be expressed in terms of nanometers, or materials composed therefrom. For example, a nanoscale structure may refer to structures having dimensions of greater than 0 nm to about 999 nm, greater than 0 nm to about 500 nm, greater than 0 to about 100 nm, greater than 0 to about 50 nm, about 20 to about 50 nm, about 10 to about 20 nm, about 5 to about 10 nm, about 1 to about 5 nm, about 1 nm, or about 0.1 to about 1 nm.

“Tissue” is defined herein to refer to a group of cells with a specific function in the body of an organism. Examples of tissues found in some animals include, without limitation, skin tissue, gingival tissue, corneal tissue, lung tissue, vascular tissues, bone, and muscle tissue. Tissues are usually composed of nearly identical cells and the intercellular substances surrounding them, and often are organized into larger units called organs.

In accordance with the present invention there may be numerous tools and techniques within the skill of the art, such as those commonly used in molecular immunology, cellular immunology. pharmacology, microbiology, molecular biology, biochemistry, protein chemistry, and cell biology. See, e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N J; Bonifacino et al. eds. (2005) Current Protocols in Cell Biology. John Wiley and Sons, Inc.: Hoboken, N J; Coligan et al. eds. (2005) Current Protocols in Immunology, John Wiley and Sons, Inc.: Hoboken, N J; Coico et al. eds. (2005) Current Protocols in Microbiology, John Wiley and Sons, Inc.: Hoboken, N J; Coligan et al. eds. (2005) Current Protocols in Protein Science, John Wiley and Sons, Inc.: Hoboken, N J; and Enna et al. eds. (2005) Current Protocols in Pharmacology, John Wiley and Sons, Inc.: Hoboken, N J.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Preparation of Calreticulin

Methods for the preparation and analysis of calreticulin, such as tissue extraction, recombinant protein technology in bacteria or yeast, anion and cation exchange and hydrophobic interaction chromatography, alcohol precipitation, cellulose acetate electrophoresis, polyacrylamide gel electrophoresis (PAGE), measurement of protein concentration, and microanalysis of SDS-PAGE electroblotted protein reverse phase HPLC, mass spectrometry, are known in the art and are described in detail in U.S. Pat. No. 5,591,716, which is incorporated herein by reference in its entirety.

As a consequence of the process of producing proteins in bacteria, recombinant calreticulin is produced with amino acid residues at the N-terminus that are not present in natural calreticulin. The additional N-terminal residues do not interfere with the beneficial effects of calreticulin on chronic wound healing. Examples of calreticulin molecules that may be used in the polymeric matrices of the present disclosure include the following, or a variant thereof:
(a) Recombinant human calreticulin having an N-terminus with an added histidine tag and two additional amino acids (GenWay Biotech, Inc., San Diego C A) (“GenWay CRT”). The histidine tag aids in the purification of the calreticulin on a nickel-Sepharose affinity column. The two additional amino acid residues between the N-terminal start methionine of calreticulin are glutamate and phenylalanine. The N-terminus of this “calreticulin+2 amino acids” has the amino acid sequence MHHHHHHHHEF (SEQ ID NO:3).
(b) Recombinant rabbit and human calreticulin having a histidine tag and five additional amino acids at the N-terminus of the natural rabbit and human CRT amino acid sequence. Thus, one such recombinant calreticulin has a histidine tag and five additional amino acids at the N-terminus of the natural rabbit CRT, and another such recombinant calreticulin has a histidine tag and five additional amino acids at the N-terminus of natural human CRT. The additional amino acids are of the gene III sequence in the pBAD plasmid, which is used to direct calreticulin protein to the periplasmic space of E. coli for ease of isolation. The gene III sequence is 23 amino acids. The gene III sequence is cleaved by the E. coli to produce a CRT with 5 amino acids at the N-terminus. This CRT+his tag+5 amino acids molecule is referred to herein as “Michalak 5 CRT+tag.” The Michalak 5 CRT N-terminus has the amino acid sequence MHHHHHHHHTMELE (SEQ ID NO:4). Natural (non-recombinant) human calreticulin has the amino acid sequence represented in SEQ ID NO:1. The amino acid sequence for natural rabbit calreticulin is represented by SEQ ID NO:7.
(c) Recombinant rabbit and human calreticulin having a histidine tag and 23 additional amino acids at the N-terminus of the natural rabbit (SEQ ID NO:7) and human (SEQ ID NO:1) CRT amino acid sequence. Thus, one such recombinant calreticulin has a histidine tag and 23 additional amino acids at the N-terminus of the natural rabbit CRT, and another such recombinant calreticulin has a histidine tag and 23 additional amino acids at the N-terminus of natural human CRT. This CRT+his tag+23 amino acids molecule is referred to herein as “Michalak 23 CRT+tag.” The Michalak 23 CRT N-terminus has the amino acid sequence MHHHHHHHHMKKLLFAIPLVVPFYSHSTMELE (SEQ ID NO:5),
(d) Recombinant rabbit and human calreticulin having five additional amino acids at the N-terminus of the natural rabbit and human CRT amino acid sequence. Thus, one such recombinant calreticulin has five additional amino acids at the N-terminus of the natural rabbit CRT, and another such recombinant calreticulin has five additional amino acids at the N-terminus of natural human CRT. The additional amino acids are of the gene III sequence in the pBAD plasmid, which is used to direct calreticulin protein to the periplasmic space of E. coli for ease of isolation. This CRT+his tag+5 amino acids molecule is referred to herein as “Michalak 5 CRT.” The Michalak 5 CRT N-terminus has the amino acid sequence TMELE (SEQ ID NO:8). Natural (non-recombinant) human calreticulin has the amino acid sequence represented in SEQ ID NO:1. The amino acid sequence for natural rabbit calreticulin is represented by SEQ ID NO:7.
(e) Recombinant rabbit and human calreticulin having 23 additional amino acids at the N-terminus of the natural rabbit (SEQ ID NO:7) and human (SEQ ID NO:1) CRT amino acid sequence. Thus, one such recombinant calreticulin has 23 additional amino acids at the N-terminus of the natural rabbit CRT, and another such recombinant calreticulin has 23 additional amino acids at the N-terminus of natural human CRT. This CRT+23 amino acids molecule is referred to herein as “Michalak 23 CRT.” The Michalak 23 CRT N-terminus has the amino acid sequence MKKLLFAIPLVVPFYSHSTMELE(SEQ ID NO:9),
(f) Natural dog pancreas calreticulin (“NAT-CRT”). The amino acid sequence of NAT-CRT is represented by SEQ ID NO:6; and
(g) Recombinant human calreticulin having no additional amino acids at the N-terminus and also lacking the signal sequence (first 17 amino acids) and starting with EPAV (Glu, Pro, Ala, Val) (SEQ ID NO: 11). This calreticulin protein can be produced from yeast using methods described in Čiplys et al., 2015 and US Pat. No. 9,796,971, both of which are incorporated herein by reference in their entireties.

The present invention encompasses calreticulin peptide fragments and other functional derivatives of calreticulin which have the functional activity of promoting healing of a chronic wound or the function of affecting a process associated with enhancing acute wound healing and chronic or impaired wound healing or tissue repair.

In an embodiment, “functional derivatives” of calreticulin are used in the polymeric matrices of the present disclosure. By “functional derivative” is meant a “fragment,” “variant,” “analog,” or “chemical derivative” of calreticulin. A functional derivative retains at least a portion of the function of calreticulin, such as upregulating TGF-β3 expression in skin, inducing cell migration, stimulating cell proliferation, inducing extracellular matrix and integrin, laminin-5, p-FAK, and cytoskeletal proteins, which permits its utility in accordance with the present invention. A “fragment” of calreticulin refers to any subset of the molecule, that is, a shorter peptide. A “variant” of calreticulin refers to a molecule substantially similar to either the entire protein or a fragment thereof. Variant peptides may be conveniently prepared by direct chemical synthesis of the variant peptide or producing the peptide by genetic recombinant technology, using methods well-known in the art.

It will be understood that the protein useful in the methods and compositions of the present invention can be biochemically purified from a cell or tissue source. For preparation of naturally occurring calreticulin, any of a number of tissues of adult or of fetal origin can be used. Because the gene encoding human calreticulin is known (GenBank Accession No. NC_000019.8, (SEQ ID NO: 2); Fliegel et al., supra; Baksh et al., (1991) supra; Rokeach et al., supra, Baksh et al. (1992) supra, Michalak et al., (1992), supra); McCauliffe et al., J Clin Invest. 1990;86:332) and can be isolated or synthesized, the polypeptide can be synthesized substantially free of other proteins or glycoproteins of mammalian origin in a prokaryotic organism, in a non-mammalian eukaryotic organism, by a yeast, or by a baculovirus system, if desired. Alternatively, methods are well known for the synthesis of polypeptides of desired sequence on solid phase supports and their subsequent separation from the support.

Alternatively, amino acid sequence variants of the protein or peptide can be prepared by mutations in the DNA which encodes the synthesized peptide. Such variants include, for example, deletions from, or insertions or substitutions of, residues within the amino acid sequence. Any combination of deletion, insertion, and substitution may also be made to arrive at the final construct, provided that the final construct possesses the desired functional activity. Obviously, the mutations that will be made in the DNA encoding the variant peptide must not alter the reading frame and preferably will not create complementary regions that could produce secondary mRNA structure (see European Patent Publication No. EP 75,444).

At the genetic level, these variants ordinarily are prepared by site-directed mutagenesis (as exemplified by Adelman et al., DNA 2:183 (1983) of nucleotides in the DNA encoding the calreticulin protein or a peptide fragment thereof, thereby producing DNA encoding the variant, and thereafter expressing the DNA (cDNA, RNA, and protein) in recombinant cell culture (see below). The variants typically exhibit the same qualitative biological activity as the nonvariant peptide.

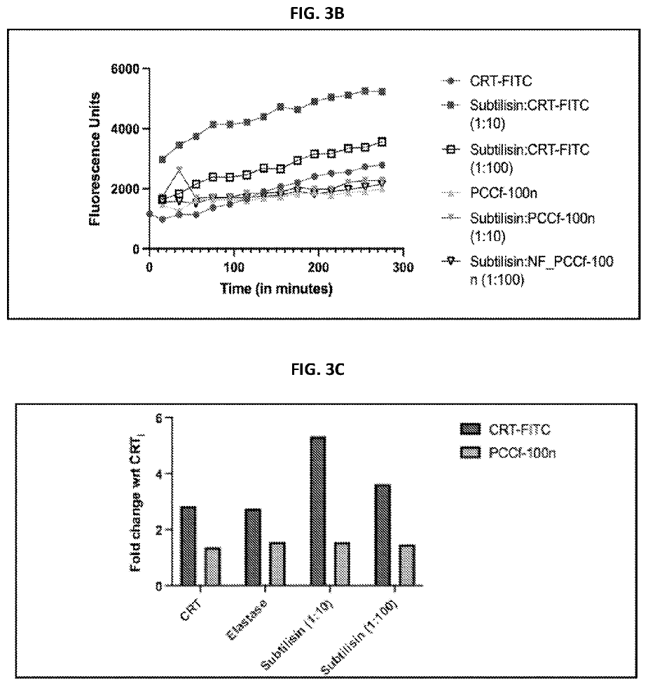

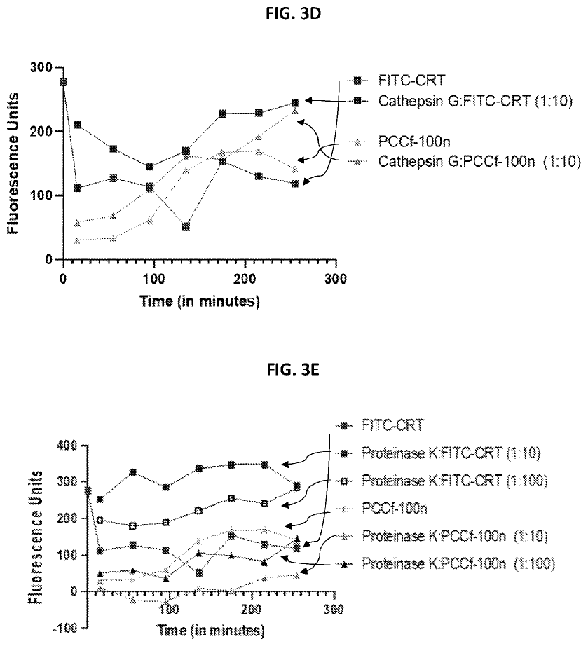

A preferred group of variants of calreticulin are those in which at least one amino acid residue in the protein or in a peptide fragment thereof, and preferably, only one, has been removed and a different residue inserted in its place. For a detailed description of protein chemistry and structure, see Schulz, G. E. et al., PRINCIPLES OF PROTEIN STRUCTURE, Springer-Verlag, New York, 1978, and Creighton, T. E., PROTEINS: STRUCTURE AND MOLECULAR PROPERTIES, W. H. Freeman & Co., San Francisco, 1983, which are hereby incorporated by reference. The types of substitutions which may be made in the protein or peptide molecule described herein may be based on analysis of the frequencies of amino acid changes between a homologous protein of different species, such as those presented in Table 1-2 of Schulz et al. (supra) and FIGS. 3-9 of Creighton (supra). Based on such an analysis, conservative substitutions are defined herein as exchanges within one of the following five groups.
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Note the Schulz et al. would merge Groups 1 and 2, above. Note also that Tyr, because of its hydrogen bonding potential, has some kinship with Ser, Thr, etc.

Preferred deletions and insertions, and substitutions, according to the present invention, are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays which are described in more detail below. For example, a change in the immunological character of the protein peptide molecule, such as binding to a given antibody, is measured by a competitive type immunoassay. Biological activity is screened in an appropriate bioassay, as described below.

Modifications of such peptide properties as redox or thermal stability, hydrophobicity, susceptibility to proteolytic degradation or the tendency to aggregate with carriers or into multimers are assayed by methods well known to the ordinarily skilled artisan.

An “analog” of calreticulin refers to a non-natural molecule substantially similar to either the entire molecule or a fragment thereof.

A “chemical derivative” of calreticulin contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

Additionally, modified amino acids or chemical derivatives of amino acids of calreticulin or fragments thereof, according to the present disclosure may be provided, which polypeptides contain additional chemical moieties or modified amino acids not normally a part of the protein. Covalent modifications of the peptide are thus included within the scope of the present disclosure. The following examples of chemical derivatives are provided by way of illustration and not by way of limitation.

Aromatic amino acids may be replaced with D-or L-naphthylalanine, D- or L-phenylglycine, D- or L-2-thienylalanine, D- or L-1-, 2-, 3- or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D-or L-p-biphenylphenylalanine, D-or L-p-methoxybiphenylphenylalanine, D- or L-2-indole(alkyl)alanine, and D- or L-alkylalanine where alkyl may be substituted or unsubstituted methyl, ethyl, propyl, hexyl, butyl, pentyl, isopropyl, isobutyl, sec-isotyl, isopentyl, non-acidic amino acids, of chain lengths of C1-C20.

Acidic amino acids can be substituted with non-carboxylate amino acids while maintaining a negative charge, and derivatives or analogs thereof, such as the non-limiting examples of (phosphono)-alanine, glycine, leucine, isoleucine, threonine, or serine; or sulfated (for example,—SO₃H) threonine, serine, tyrosine.

Other substitutions may include unnatural hydroxylated amino acids may made by combining “alkyl” with any natural amino acid. Basic amino acids may be substituted with alkyl groups at any position of the naturally occurring amino acids lysine, arginine, ornithine, citrulline, or (guanidino)-acetic acid, or other (guanidino) alkyl-acetic acids, where “alkyl” is defined as above. Nitrile derivatives (for example, containing the CN-moiety in place of COOH) may also be substituted for asparagine or glutamine, and methionine sulfoxide may be substituted for methionine.

In addition, any amide linkage the polypeptides can be replaced by a ketomethylene moiety. Such derivatives are expected to have the property of increased stability to degradation by enzymes, and therefore possess advantages for the formulation of compounds which may have increased in vivo half lives, as administered by various routes as described herein.

In addition, any amino acid representing a component of the peptides can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives have the property of greatly increased stability to degradation by enzymes, and therefore are advantageous in the formulation of compounds which may have longer in vivo half lives, when administered by various routes.

Additional amino acid modifications in calreticulin or in a peptide thereof may include the following.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-13-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides, which reverses the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; 0-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine c-amino group.

The specific modification of tyrosyl residues has been studied extensively with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimides (R′-N-C-N-R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are deamidated to the corresponding glutamyl and aspartyl residues. Alternatively, these residues are deamidated under mildly acidic conditions. Either form of these residues falls within the scope of this invention.

Derivatization with bifunctional agents is useful for cross-linking the peptide to a water-insoluble support matrix or to other macromolecular carriers. Commonly used cross-linking agents include, e.g., 1, 1-bis(diazoacety 1)-2-phenylethane, glutaraldehyde, N-hydroxy succinimide esters, for example, esters with 4-azidosalicylic acid, homobifunctional imidoesters, including disuccinimidyl esters such as 3,3′-dithiobis-(succinimidyl-propionate), and bifunctional maleimides such as bis-N-maleimido-1,8-octane. Derivatizing agents such as methyl-3-[(p-azidophenyl)dithic]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the α-amino groups of lysine, arginine, and histidine side chains (Creighton, supra), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein and the like. Moieties capable of mediating such effects are disclosed, for example, in Remington's Pharmaceutical Sciences, 16th ed., Mack Publishing Co., Easton, Pa. (1980).

Production of Calreticulin and Fusion Proteins that Promote Wound Healing

Calreticulin may be purified from a tissue source using conventional biochemical techniques, or produced recombinantly in either prokaryotic or eukaryotic cells using methods well-known in the art. See, Sambrook, J. et al., MOLECULAR CLONING: A LABORATORY MANUAL, 2nd Edition, Cold Spring Harbor Press, Cold Spring Harbor, N. Y., 1989, which reference is hereby incorporated by reference in its entirety. Various references describing the cloning and expression of calreticulin have been noted above.

Fusion proteins representing different polypeptide regions in calreticulin may be used to identify regions of the protein that have the desired functional activity (binding, stimulating wound healing, specific functions associated with wound healing, etc.). When combined with the polymerase chain reaction (PCR) method, it is possible to express in bacteria or any other expression vector not limited to or as an example, yeast, eucaryotic cells, nearly any selected region of the protein.

Calreticulin, a fragment peptide thereof, or a fusion protein thereof may also be expressed in insect cells using baculovirus expression system. Production of calreticulin or functional derivatives thereof, including fusion proteins, in insects can be achieved, for example, by infecting the insect host with a baculovirus engineered to express calreticulin by methods known to those of skill. Thus, in one embodiment, sequences encoding calreticulin may be operably linked to the regulatory regions of the viral polyhedrin protein. See. Jasny, 1987, Science 238:1653. Infected with the recombinant baculovirus, cultured insect cells, or the live insects themselves, can produce the calreticulin or functional derivative protein in amounts as great as 20 to 50% of total protein production. When live insects are to be used, caterpillars are presently preferred hosts for large scale production according to the invention.

Fragments of calreticulin are purified by conventional affinity chromatography using antibodies, preferably monoclonal antibodies (mAbs) that recognize the appropriate regions of calreticulin. The mAbs specific for the most highly conserved regions in calreticulin can be used to purify calreticulin protein from mixtures. Also, the fragments can be his-tagged and purified by Nickel-Speharose or purified by chromatographic means or isolated by common chromatographic methods based in protein isolation by charge, hydrophobicity, or molecular mass.

### Routes of Administration and Dosages

The preferred animal subject of the present disclosure is a mammal. By the term “mammal” is meant an individual belonging to the class Mammalia. The invention is particularly useful in the treatment of human subjects.

The present disclosure provides for methods of treatment of wounds, which methods comprise applying a polymeric matrix described herein to the wound in the subject. The disorders that may be treated according to this disclosure include, but are not limited to acute wounds, chronic wounds, corneal wounds, bone and cartilage repair, injury due to surgical procedures, wrinkles, and alopecia as well as other uses of calreticulin disclosed herein.

In some embodiments, the chronic wound is a chronic diabetic wound, a venous or arterial stasis ulcer, a pressure ulcer, or an ulcer resulting from sickle cell disease (SCU). In some embodiments, the chronic diabetic wound is diabetic foot ulcer (DFU). In some embodiments, the DFU is on the plantar surface of the foot.

In some embodiments, the wound is a post-surgical wound or an internal wound.

In some embodiments, the wound is a corneal wound. For eye application, the polymeric matrix may comprise a polymer such as hyaluronic acid, and/or be combined with fibronectin or other extracellular matrix proteins such as collagen. In some embodiments, the extracellular matrix proteins can be incorporated by electrospinning or other means to produce a scaffold that is incorporated with calreticulin. Also, cytokines or interleukins such as IL-1 can be combined in these embodiments.

For the topical applications, it is preferred to administer an effective amount of a composition according to the present invention to an affected wound area, in particular the skin surface and/or inner wound bed. This amount of calreticulin can generally range from about 1 pg to about 1 g per application, depending upon the area to be treated, the severity of the symptoms, and the nature of the topical vehicle employed. The dosage of the therapeutic formulation may vary widely, depending upon the size of the wound, the patient's medical history, the frequency of administration, the manner of administration, the clearance of the agent from the host, and the like. The dose may be administered with each wound dressing change. The dose may be administered once daily, more than once daily, or as infrequently as weekly or biweekly.

A polymeric matrix of the present disclosure can include any pharmaceutically acceptable carrier or excipient. The term “carrier” refers to a diluent, adjuvant, excipient, or vehicle with which the compound is administered. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. Pharmaceutically acceptable carriers can be sterile liquids, such as water and oils, including those of petroleum, animal, vegetable or synthetic origin, such as peanut oil, soybean oil, mineral oil, sesame oil and the like. Others are gels, such as hydrogels, hyaluronic acid (HA), collagen, materials consisting of naturally occurring or synthetic substances, or any other matrix protein such as perlecan, proteoglycans, glycoaminoglycans, fibrin gels, and polymers. Other suitable carriers include micelles and liposomes. Suitable pharmaceutical carriers are further described in “Remington's Pharmaceutical Sciences” by E. W. Martin.

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol. 1: Principles and Practice, which is incorporated herein by reference to the extent of teaching such derivatives. Preferred pharmaceutically acceptable derivatives are salts, solvates, esters, carbamates, and phosphate esters. Particularly preferred pharmaceutically acceptable derivatives are salts, solvates, and esters. Most preferred pharmaceutically acceptable derivatives are salts and esters.

In an embodiment, peptide sequences from calreticulin are inserted into or used to replace sequences within “scaffold” proteins which can be incorporated into a polymeric matrix of the present disclosure. Accordingly, a “scaffold protein” of the present invention is a protein which includes a functional calreticulin sequence, either as an inserted sequence or as a replacement sequence for a homologous (corresponding) sequence of the scaffold protein. The scaffold protein adopts a native conformation. The calreticulin and scaffold can alternate positions; these terms are used to indicate the source of sequences introduced into the “scaffold.”

The administration route may be any mode of administration known in the art, including but not limited to topically or internally. The present invention also provides pharmaceutical and cosmetic compositions comprising an amount of calreticulin, or a functional derivative or fragment thereof, effective to promote the healing of a wound or exert any other therapeutic or cosmetic effect relevant for the present invention, in a pharmaceutically or cosmetically acceptable carrier.

The pharmaceutical composition of the present invention is preferably applied to site of action (e.g., topically, subcutaneously [e.g., by injection], intradermally, transdermally [e.g., by transdermal patch], or via intracorporal application during surgery).

For topical application, polymeric matrices of the present disclosure may also incorporate any topically applied vehicles such as salves, ointments, or liposomes, which have both a soothing effect on the skin as well as the ability to facilitate administration of the active ingredient directly to the affected area. Example vehicles for topical preparations include ointment bases, e.g., polyethylene glycol-1000 (PEG-1000); conventional creams such as HEB or HEC cream; gels; hydrox ethyl cellulalose; as well as petroleum jelly and the like. Another example vehicle is a petrolatum/lanolin vehicle. Yet another example vehicle is hydrogels, for examples, as described in U.S. Pat. Nos. 7,790,194, 8,465,779, 8,231,890, and in U.S. Publication No. US 2014/0228453.

The dosage administered will be dependent upon the age, health, and weight of the recipient, kind of concurrent treatment, if any, frequency of treatment, and the nature of the effect desired.

Effective doses of calreticulin included in a polymeric matrix of the present disclosure for therapeutic uses discussed above may be determined using methods known to one skilled in the art. Effective doses may be determined, preferably in vitro, in order to identify the optimal dose range using any of the various methods described herein. In one embodiment, an aqueous solution of a calreticulin protein or peptide is administered by intravenous injection. Each dose may range from about 1 pg/kg body weight to about 100 mg/kg body weight, e.g., from about 0.001 μg/kg to 10 mg/kg body weight or from about 0.1 μg/kg to 10 mg/kg body weight. The dosing schedule may vary from one time only to once a week to daily or twice (or more) daily depending on a number of clinical factors, including the type of wound, its severity, and the subject's sensitivity to the protein. Calreticulin would likely not be immunogenic since it is present in every cell in the human body. Non-limiting examples of dosing schedules are 3 μg/kg administered twice a week, three times a week or daily; a dose of 7 μg/kg twice a week, three times a week or daily; a dose of 10 μg/kg twice a week, three times a week or daily; or a dose of 30 μg/kg twice a week, three times a week or daily. In the case of a more severe chronic wound, it may be preferable to administer doses such as those described above by alternate routes, including intramuscularly, intraperitoneally or intrathecally. Continuous infusion may also be appropriate.

Polymeric matrices of the present disclosure may include or be administered in combination with an effective amount of at least one other agent that is, itself, capable of promoting the healing of wounds or treating accompanying symptoms. Such agents include growth factors, anti-infectives, including anti-bacterial, anti-viral and anti-fungal agents, local anesthetics, and analgesics, collagens, fibrin gels, heat shock proteins, glycosaminoglycans (e.g., hyaluronic acid), proteoglycans (e.g., perlecan, heparin sulfate), glycoproteins (e.g., fibronectin), syndecan, gelatin, suitable chemical or natural polymers, or a combination thereof. Other agents that can be applied to a wound include but, are not limited to, calreticulin as part of a living skin substitute (skin device) or a synthetic, chemical or natural scaffold or matrix or polymer or augraft or allograft, thereof.

Combination treatment according to the present invention includes applying a polymeric matrices of the present disclosure and one or more additional agent in the same or separate dosage forms. Such additional agents include, among others, agents which are known to promote wound healing or to treat problems or symptoms associated with chronic wounds. Examples of such agents include hyaluronic acid, disinfectants such as antibacterial agents or antiviral agents, anti-fungal agents, anti-inflammatory agents, agents which induce relief from pain or itching, and the like. Also included are growth factors which promote wound healing, including, but not limited to, transforming growth factor-α, transforming growth factor-β, fibroblast growth factor-α, fibroblast growth factor-β, FGFs in general, epidermal growth factor, platelet-derived growth factor, endothelial cell-derived growth factor, insulin-like growth factors, vascular endothelial growth factors (VEGF), and granulocyte colony-stimulating factor. In accordance with the methods of the present disclosure, polymeric matrices of the present disclosure applied in combination with an additional agent includes any overlapping or sequential application of the polymeric matrix and the additional agent. Thus, for example, methods according to the present invention encompass applying a polymeric matrix and an additional agent simultaneously or non-simultaneously.

Further, according to the present invention, polymeric matrices of the present disclosure and an additional agent can be administered by the same route (e.g., both are administered topically) or by different routes (e.g., a polymeric matrix is administered topically and an additional agent is administered orally).

The pharmaceutical compositions of the present invention may be administered by any means that achieve their intended purpose. Amounts and regimens for the administration of calreticulin, or a derivative thereof, can be determined readily by those with ordinary skill in the clinical art of treating wounds.

### EXAMPLES

The following examples are provided to further describe some of the embodiments disclosed herein. The examples are intended to illustrate, not to limit, the disclosed embodiments.

### Example 1. Fabrication of Calreticulin (CRT)-containing Polycaprolactone (PCL)/type I Collagen (Col1) Nanofibers (NFs)

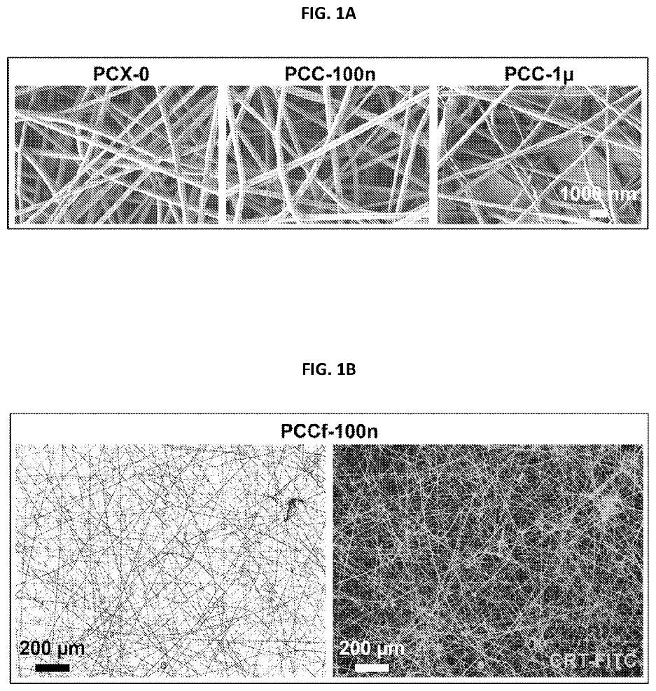

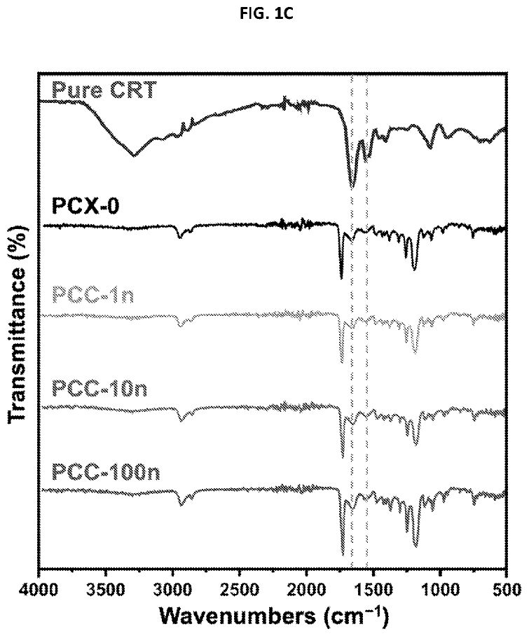

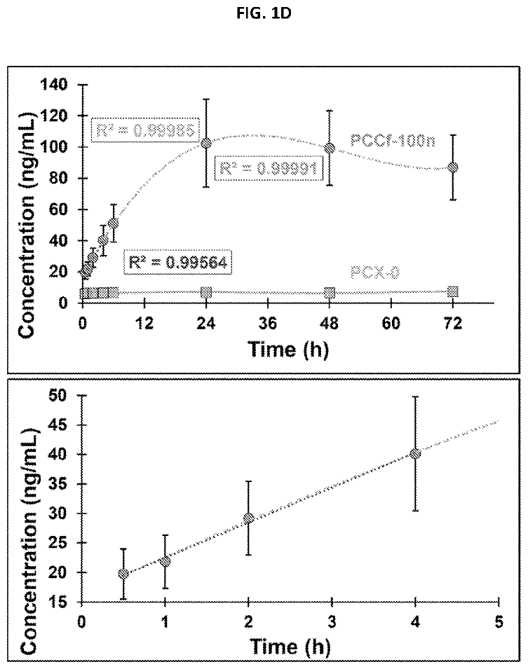

CRT-containing PCL/Col1 NFs were fabricated as described in the Materials and Methods section below. Successful incorporation of CRT into PCL/Col1 NFs was confirmed using attenuated total reflectance-fourier transform infrared (ATR-FTIR) spectroscopy, in which semi-quantification of signature absorption peaks showed a correlation with increasing concentrations of CRT (FIG. 1C). SEM examination (FIG. 1A) revealed a comparable fiber diameter (Table 1) and organization despite addition and increasing concentrations of CRT at least up to PCC-100n conditions [322±80 nm (PCX-0) vs. 331±80 nm (PCC-100n), p=0.57 by a two-sided t-test assuming unequal variances]. However, at a ten-fold higher concentration (PCC-1 μ), a noticeable change in fiber diameter was seen (p<1×10⁻⁸ by single factor analysis of variance (ANOVA), yielding smaller fibers with larger variability when using the same electrospinning parameters (FIG. 1A). To better visualize CRT distribution in NFs, CRT-FITC was used to fabricate PCCf-100n NFs. As shown in FIG. 1B, CRT-FITC was distributed uniformly throughout the NFs, suggesting CRT was well dispersed in the electrospinning solution prior to the formation of NFs.

TABLE 1
Fiber diameters measured from SEM images.

| Fiber Type | Measured Diameter (nm) |
|---|---|
| PCX-0 | 322 ± 80 |
| PCC-10n | 348 ± 62 |
| PCC-100n | 331 ± 80 |
| PCC-1μ | 245 ± 94 |

CRT, calreticulin;
PCL, polycaprolactone;
Col1, type I collagen;
PCX, PCL/Col1;
PCC, PCL/Col1/CRT

### Example 2. Sustained Release of CRT From CRT-containing PCL/Col1 NFs

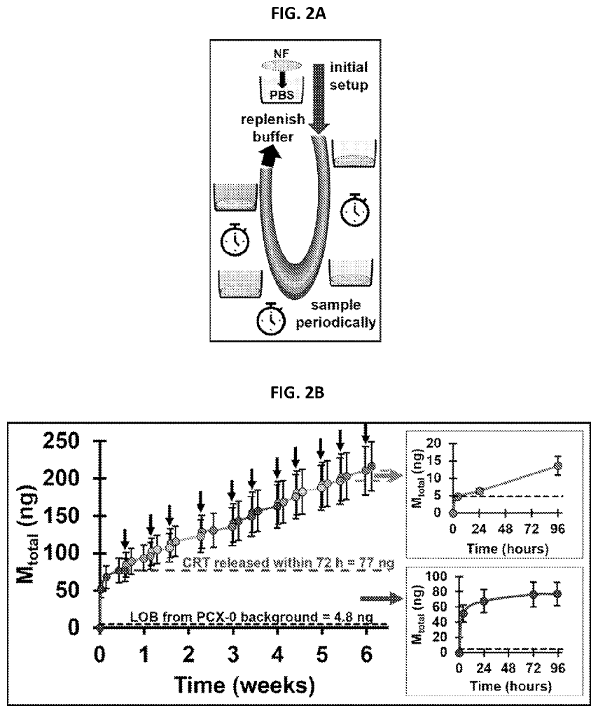

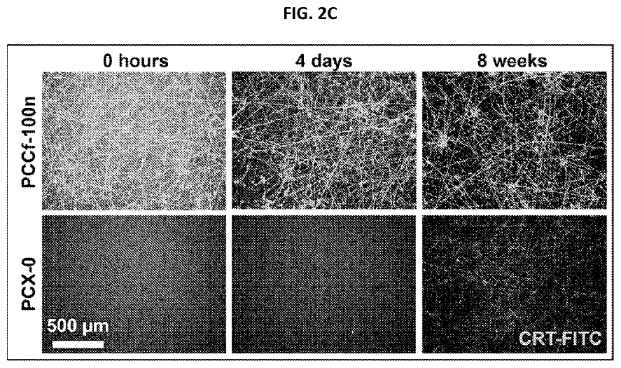

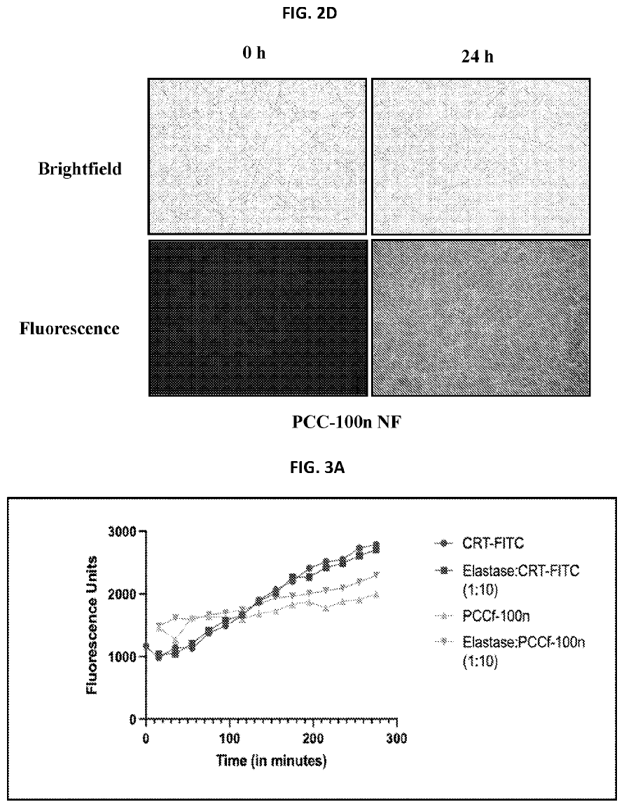

An initial release experiment was performed at 37° C. with shaking at 80 rpm to determine sufficient sampling timepoints to establish the release profile (FIG. 1D). More appropriate release experiments were conducted at 37° C. under static conditions (i.e., without shaking) to better emulate the circumstances of in vitro cell culture and in vivo topical application. Within each round of the prolonged release study, samples were collected and replaced with PBS (70 μL) at 4 h, 24 h, and 48-120 h following complete removal and PBS replenishment (700 μL), with 2-5 day-long rounds repeating 11 times over a span of 6 weeks (FIGS. 2A and 2B). As noted, a significant amount of CRT-FITC was burst released within 4 h (52±11 ng) followed by a slower release during the first round (bottom inset in FIG. 2B), with 77±16 ng of CRT-FITC released within the first 72 h and no further release observed thereafter (p=0.19 by paired t-test) until replenishment with fresh PBS (i.e., starting a new “round” of release). During every subsequent round, smaller amounts of CRT-FITC (9-19 ng) were released from the PCCf-100n NFs, with a total of 210±32 ng released after 11 rounds of PBS replenishment. Importantly, the smaller amounts of CRT-FITC released during rounds 2 -11 remained distinguishable above background levels, as demonstrated for “round 11” in the top inset of FIG. 2B To determine this background, PCX-0 NF samples were subjected to the same release experiment at the same time, and the baseline in all graphs of FIG. 2B (4.8±0.9 ng) represents the average false mass calculated from PCX-0 samples across all 11 rounds of the prolonged release study independently, akin to a limit of blank (LOB).³⁵ To visually confirm the CRT-FITC release, fluorescence images were taken with PCCf-100n NFs throughout the release experiment (FIG. 2C). The fluorescence signal decreased in the PCCf-100n group yet remained clearly distinguishable even after 8 weeks. Interestingly, after 8 weeks submerged in PBS, very faint fluorescence was seen with PCX-0 NFs, most likely due to the autofluorescence of Col1.

### Example 3. Resistance to Proteolytic Degradation of CRT in CRT-containing PCL/Col1 NFs

To determine whether CRT electrospun into NFs is putatively protected from proteolytic degradation compared with CRT without NFs (i.e., free CRT), elastase, subtilisin, cathepsin G, and proteinase K were separately incubated with CRT-FITC or PCCf-100n NFs and protein stability was assessed by measuring the fluorescence intensity of the solution or supernatant respectively. As shown in FIG. 3A, over a period of ˜4.5 h, a similar incremental increase in fluorescence was observed for untreated free CRT-FITC in PBS compared to elastase-treated free CRT-FITC. both showing a 2.8-fold change in fluorescence units (FIG. 3C). However, the relative increase in fluorescence intensity over time following elastase treatment was less for the CRT-FITC sequestered in NFs at 1.6-fold [with elastase] compared to 1.4-fold [without elastase]. Treatment of CRT-FITC with subtilisin at a ratio of 1:10 and 1:100 (enzyme to protein) gave a dose-dependent increase in fluorescence intensity (FIG. 3B) with a remarkable fold-change of 5.3 at the 1:10 dilution and 3.6 at 1:100 (FIG. 3C). Interestingly, as with elastase, there was a consistent unchanged fluorescence intensity in the PCCf-100n NFs in the presence or absence of subtilisin during the incubation period. PCCf-100n NFs treated with subtilisin at 1:10 and 1:100 ratios showed a fold change of 1.6 and 1.5 respectively (FIG. 3C). Therefore, the data suggest that CRT sequestered in NFs (PCCf-100n) is well protected from proteolytic digestion within a 4.5 h digestion period by subtilisin, an enzyme that would be high in an infected wound. Moreover, within the NFs, even at a 10 times higher concentration of subtilisin, CRT was nearly equally stabilized. Furthermore, incubation of PCCf-100n and CRT-FITC separately with cathepsin G, serine protease released by immune cells in the wound, and proteinase K, a proteolytic enzyme with broad substrate specificity, gave similar results such that CRT sequestered in the NFs (PCCf-100n) showed lower relative fluorescence units over time upon enzyme exposure than an equal amount of CRT-FITC free in solution (FIGS. 3D-3E). As shown in FIGS. 3D-3E. by fluorescence intensity (units), cathepsin G increased fluorescence intensity of calreticulin (FITC-CRT) in solution whereas calreticulin electrospun in nanofibers (PCCf-100n) showed less fluorescence both in the presence of absence of cathepsin G. Proteolytic digestion by proteinase K of FITC-CRT in solution was observed at the earliest time point of 15 min with a dose-response pattern dependent on enzyme dilution. FITC-CRT in NFs (PCCf-100n) showed less fluorescence, which was similar to the FITC-CRT control without enzyme.

### Example 4. Retention of Fibroblast Response to CRT in CRT-containing PCL/Col1 NFs

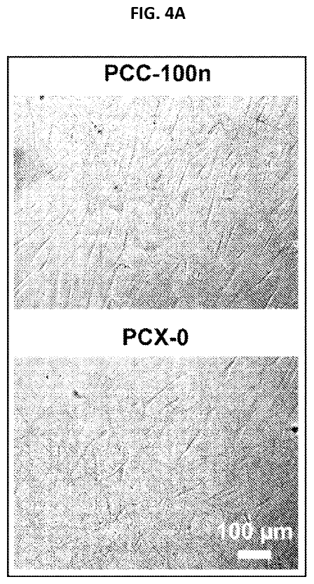

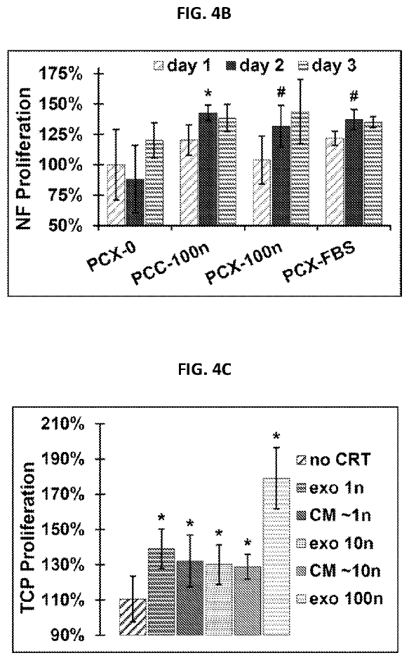

To determine whether CRT incorporated into NFs retained its ability to stimulate cell proliferation, a function important for increasing cell numbers in the wound.^{9.10} HFFs seeded onto PCC NFs were assessed for their proliferative capacity compared to those cultured on PCX-0 NFs. Examination of the culture under phase-contrast imaging revealed a higher number of HFFs on PCC-100n NFs relative to PCX-0 NFs (FIG. 4A). HFFs appeared well attached and spread on both PCC-100n and PCX-0 NFs. The observed increase in HFF proliferation was also verified using a resazurin assay (FIG. 4B). No differences existed between all groups tested on day 1 (p=0.42 by single factor ANOVA), indicating that initial cell seeding was equal such that future timepoints could be directly compared to elucidate proliferation differences. Accordingly, statistically significant differences were identified between groups on day 2: the PCC-100n group (143±7%) was significantly greater than the PCX-0 group (88±28%) (p=0.04 by one-sided t-test assuming unequal variances), and borderline significance was found when comparing the PCX-100n (132±17%) and PCX-FBS (137±8%) groups to the PCX-0 group (0.05<p<0.053 by one-sided t-tests assuming unequal variances). Interestingly, no statistical differences existed again between all groups on day 3 (p=0.38 by single factor ANOVA), suggesting that the cells had reached confluency with no further space available for the cells to be able to proliferate as contact inhibition had been reached. Confluency of the cell cultures was confirmed by subsequent immunofluorescence staining.

To demonstrate whether the CRT released from NFs exhibits a similar bioactivity to the unprocessed CRT (i.e., not electrospun into NFs and therefore without exposure to organic solvent and electric field), HFFs cultured on TCP were incubated with either conditioned media (CM) containing CRT released from NFs or media containing unprocessed CRT. Upon confirmation of comparable cell number in each well, the cells were subsequently cultured with the different media for a total of 6 days. As shown in FIG. 4C, the normalized proliferation of the negative control (“no CRT”) and positive control (“exo 100n”) were 111±13% and 179±17% respectively, and groups with either CM or exo [exogenously added] showed values between these controls: 139±11% for “exo 1n”, 132±15% for “CM˜1n”, 130±11% for “exo 10n”, and 129±7% for “CM˜10n”. Therefore, the presence of CRT in the culture media, whether unprocessed (exo) or released from NFs (CM), similarly promoted cell proliferation at equal doses compared to the cultures receiving media without CRT (p<0.05 by one-sided t-tests assuming unequal variances).

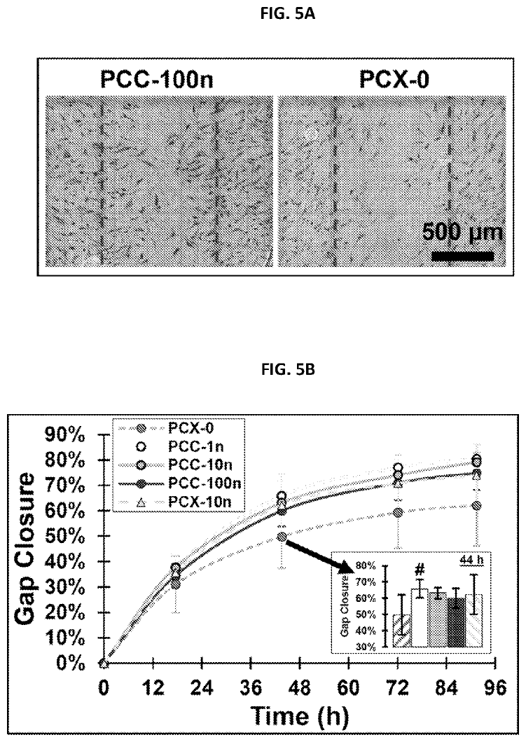

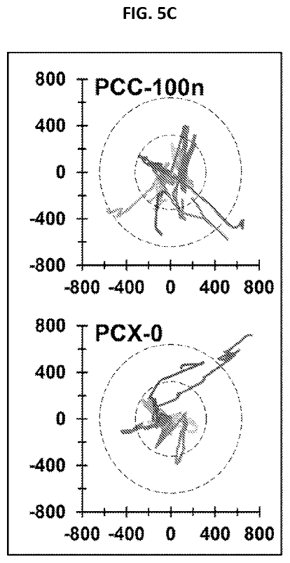

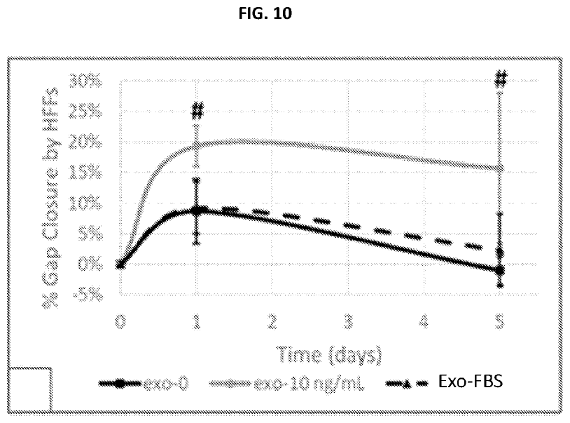

Another biological function important for wound healing is the migration of fibroblasts into the wound to therein produce extracellular matrix proteins for granulation tissue formation (neodermis). Previous studies have shown that free CRT induced both chemotaxis and motility of fibroblasts. To determine whether CRT electrospun into NFs retained the ability to mediate migration, HFFs seeded onto PCC compared to PCX-0 NFs were examined for cumulative motogenic behavior using in vitro wound-gap closure assays and for single cell directional and velocity migration using time-lapse imaging in a motility assay. In an initial experiment in which proliferation was not blocked, the PCC-100n NFs appeared to promote greater wound-gap closure compared to the PCX-0 control after 51 h (FIG. 5A). To more accurately quantify gap closure and exclude the contribution of cell proliferation, cells were fluorescently labeled with DiD prior to seeding and then pretreated with mitomycin C, an inhibitor of proliferation, after adhesion (shown in FIG. 10). FIG. 10 proves that cells induced to close the in vitro wound in response to CRT was solely due to migration. The number of cells in the gap were induced by CRT to migrate and close the gap. Proliferation does not contribute to the migratory effect of CFRT. Through time-lapse fluorescence imaging, periodic gap closure was readily quantified (FIG. 5B). No statistical difference in gap closure was found between groups with or without CRT, particularly at the earliest timepoint (i.e., at 18 h: p=0.67 by single factor ANOVA). However, at later timepoints there did appear to be a trend that the presence of CRT either in NFs or exogenously supplied slightly increased cell migration, and a borderline significance was seen between PCC-In and PCX-0 groups (0.05<p<0.10 by one-sided t-tests assuming unequal variances at 44 h, 72 h, and 91 h). It is notable that the untreated control consistently showed a large standard deviation in individual experiments. However, as shown by the error bars of migration of HFFs on PCX-0, it is possible statistical significance was not attained to increased random migration of the cells on NFs without the presence of CRT. The inset of FIG. 5B highlights the gap closure of different groups at 44 h, wherein PCC-1n was 66±6% compared to 50±12% for PCX-0, and the other groups were 63±3% (PCC-10n), 60±6% (PCC-100n), and 62±12% (PCX-10n). In terms of single cell motility, time-lapse observation showed that HFFs on PCC-100n NFs were more motile than those on PCX-0 NFs (FIG. 5C).

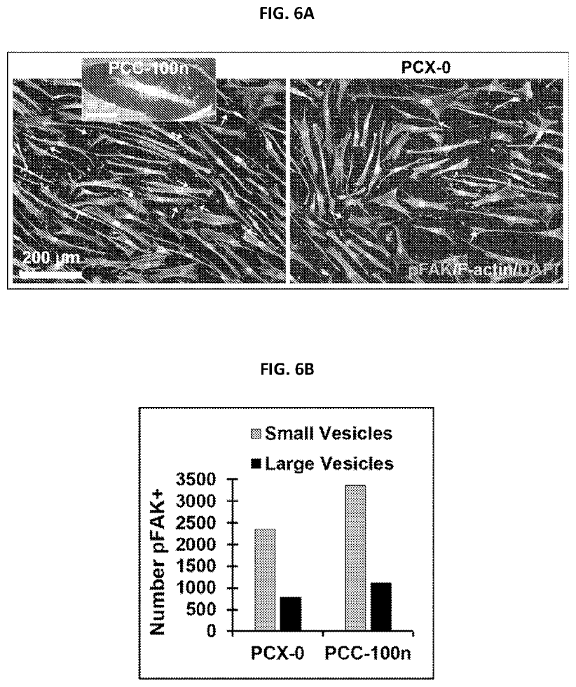

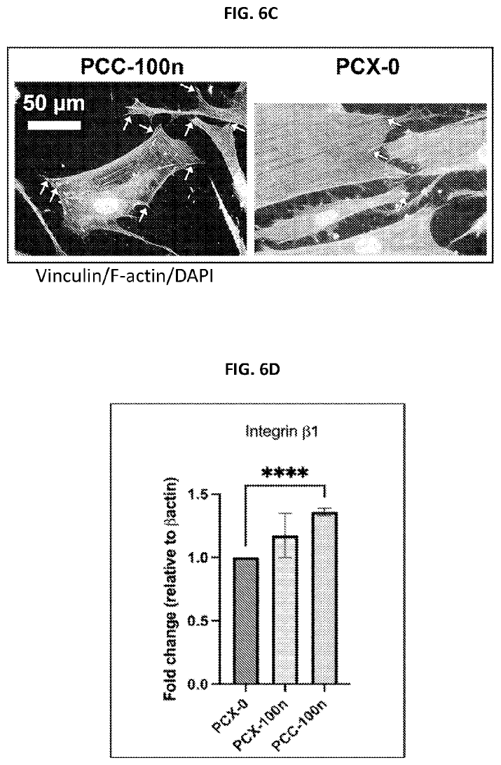

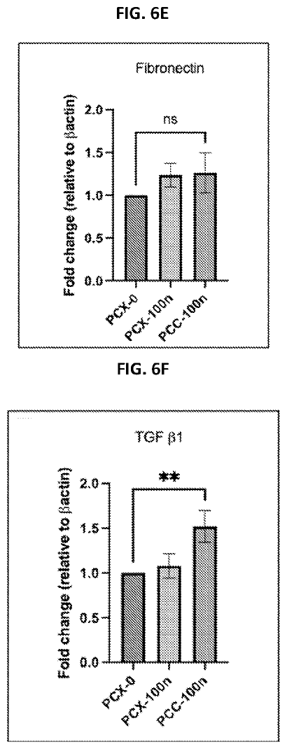

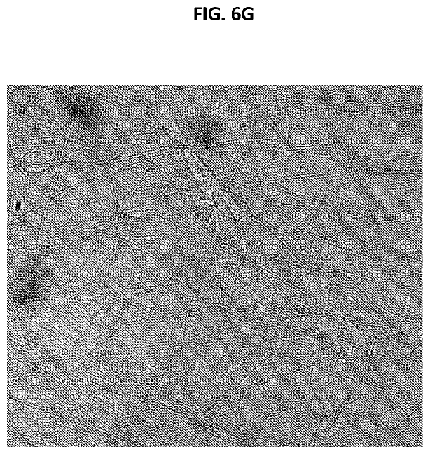

To further characterize the migratory phenotype supported by CRT electrospun into NFs, immunofluorescent staining for pFAK, vinculin, and F-actin was performed for HFFs cultured on PCX-0 and PCC-100n NFs for 24 h. As shown in FIG. 6A, pFAK (red fluorescence) was predominantly distributed in the perinuclear region of HFFs cultured on PCX-0 NFs, whereas a higher expression of pFAK was localized at the cell peripheries and, remarkably, in extracellular vesicles left behind by the cells (FIG. 6A, inset) on PCC-100n NFs. This trail of extracellular vesicles was also observed in the brightfield images of an HFF culture on PCC-10n NFs (FIG. 6G). Semi-quantification of the pFAK-enriched vesicles (red fluorescence) showed that a greater amount was present in the PCC-100n group (FIG. 6B). Although HFFs on both types of NFs were spread with an elongated morphology, cells on PCC-100n NFs appeared more uniformly stretched with more lamellipodia and filipodia than those on PCX-0 NFs (FIG. 6A). Typical vinculin (green fluorescence)-capping F-actin fibers (red fluorescence) were seen in HFFs cultured on both types of NFs but were much more pronounced on PCC-100n NFs (FIG. 6C, arrows). While evenly distributed F-actin fibers were seen across HFFs cultured on PCX-0 NFs, F-actin fibers were mainly located in the peripheral region of cells on PCC-100n NFs, consistent with a migratory cell phenotype.

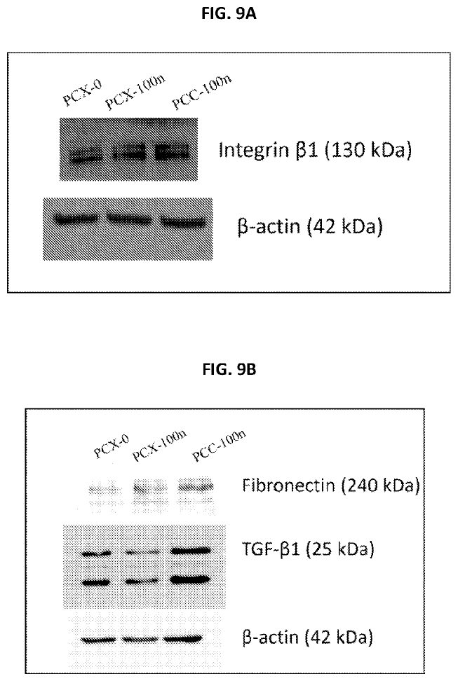

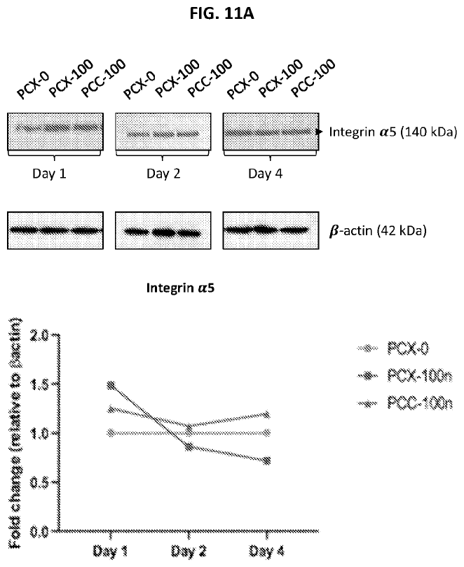

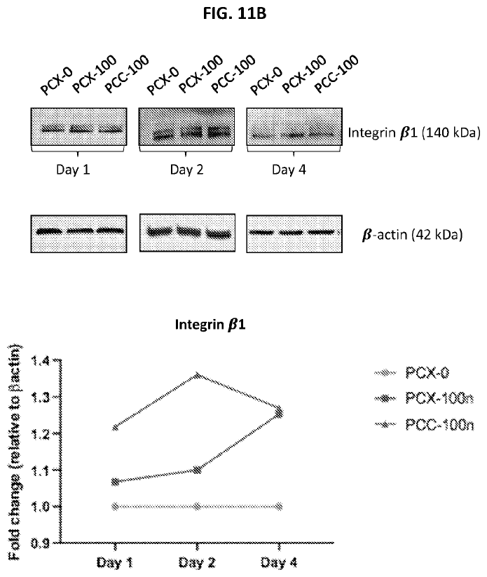

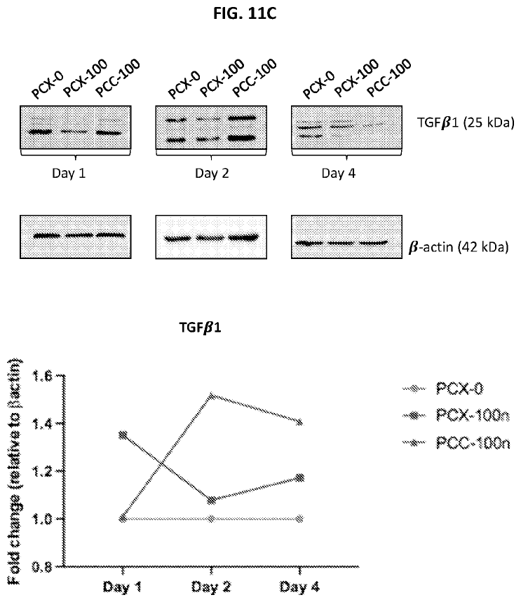

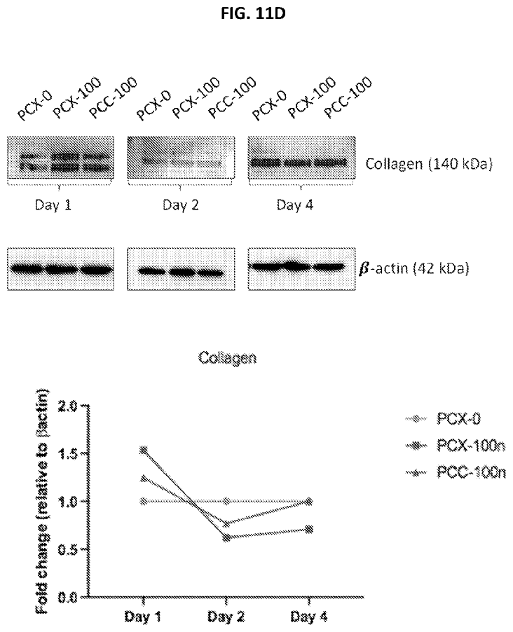

Additionally, to determine whether CRT electrospun into NFs retained the ability to induce ECM proteins and integrin β1, HFFs were cultured on NFs containing CRT (PCC-100n), NFs without CRT (PCX-0), or NFs without CRT but with CRT supplied exogenously as a control (PCX-100n) and compared for levels of integrin β1, fibronectin, and TGF-β1 (a key inducer of ECM proteins). The level of fibronectin was shown to be induced in HFFs seeded on both PCX-100n and PCC-100n NFs (FIG. 6E, FIG. 9B). However, the difference was not statistically significant as compared to PCX-0. HFFs seeded on PCC-100n and PCX-100n NFs showed an increased expression of integrin β1, which was significant at 1.4-fold higher than cells cultured on PCX-0 (FIG. 6D, FIG. 9A, FIG. 11B). HFFs seeded on PCC-100n showed a statistically significant induction of TGF-β1 as well marked by the ˜1.4-fold change as compared to both PCX-0 and PCX-100n groups as shown in FIG. 6F, FIG. 11C. These data confirm that fibroblasts (HFFs) seeded on CRT electrospun into NFs (PCC-100n) or exogenously added to NFs (PCX-100n) synthesize integrin β1 [for cell migration], fibronectin, and TGF-β1. Particularly, TGFβ1 was synthesized by cells grown on PCC-100n compared to PCX-100n and PCX-0.

### Example 5. Retention of Keratinocyte Response to CRT in CRT-containing PCL/Col1 NFs

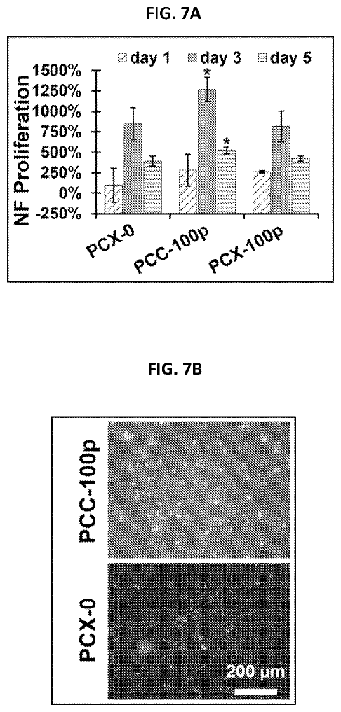

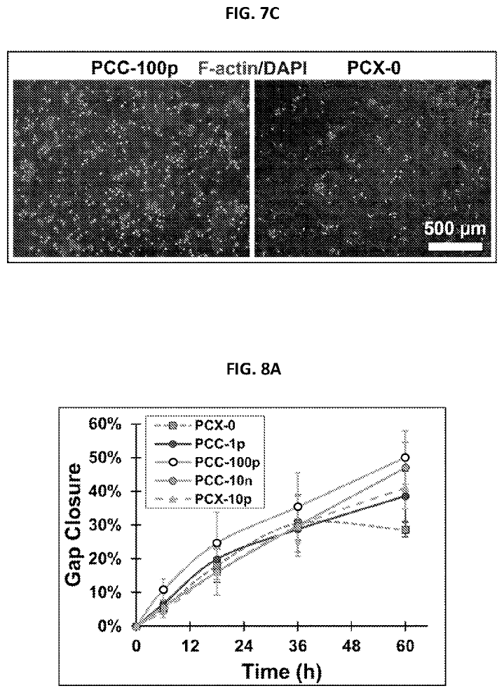

Similar to HFFs, proliferation of HEKs on various NFs was assessed by resazurin assay. As shown in FIG. 7A, no significant difference in cell proliferation (p=0.40 by single factor ANOVA) were observed on day 1 among PCX-0, PCC-100p, and PCX-100p groups. However, after culture for 3 days, proliferation on PCC-100p NFs was significantly greater than on PCX-0 NFs (1266±145% vs. 852±193%, p=0.02 by one-sided t-test assuming unequal variances). This was not true when the PCX-100p group, in which CRT was added exogenously, was compared to the PCX-0 group (817±187% vs. 852±193%, p=0.41 by one-sided t-test assuming unequal variances). Therefore, PCC-100p possessed a unique ability to stimulate proliferation of HEKs compared to exogenously added CRT (PCX-100p). Surprisingly, by day 5, the normalized metabolic activity was decreased for all the groups (PCX-0, PCC-100p, and PCX-100p reaching 393±61%, 524±39%, and 423±32% respectively) despite the statistical difference between the PCC-100p and PCX-0 groups (p=0.03 by one-sided students t-test assuming unequal variances). Cell detachment was observed in all groups, likely accounting for this decrease, which could have been caused by the long incubation with resazurin. Notably, live/dead staining showed negligible cell death suggesting loss of cell adherence To further confirm the promotion of HEK proliferation by PCC-100p NFs, immunofluorescent staining for Ki67 was performed immediately after the resazurin assay. Notably, substantially more Ki67-positive cells (green fluorescence) were found on the PCC-100p NFs than on the PCX-0 NFs (FIG. 7B) Staining for F-actin and nuclei (DAPI) confirmed more cells on PCC-100p NEs than on PCX-0 NFs (FIG. 7C).

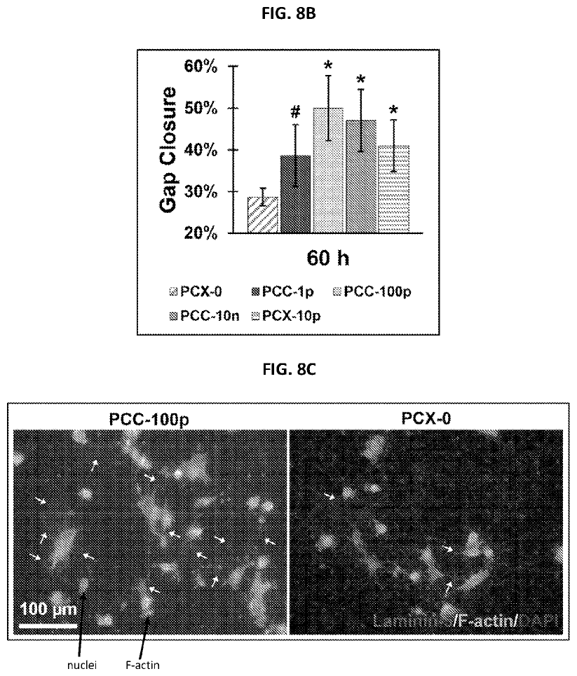

As CRT was shown to stimulate migration of keratinocytes accounting for the rapid reepithelialization in vivo, the retention for CRT electrospun into NFs to induce HEK migration was evaluated using wound-gap closure assays and immunostaining for selected migratory markers. FIG. 8A displays the quantified results of a wound healing assay in which HEKs were labeled with DiD and pretreated with mitomycin C. After 6 h, only HEKs on PCC-100p NFs showed significantly higher gap closure than those on PCX-0 NFs (p=0.05 by one-sided t-test assuming unequal variances). Throughout the course of the experiment, percent gap closure progressively increased in all groups, though it appeared to level off in the PCX-0 group at approximately 36 h. At the final timepoint examined (60 h, FIG. 8B), the presence of CRT—both incorporated in NFs (PCC groups) or supplemented exogenously into the media (PCX-10p group)—significantly promoted gap closure via cell migration relative to the PCX-0 control. A borderline significance (p=0.08 by one-sided t-test assuming unequal variances) was seen between the PCC-1p and PCX-0 groups at this timepoint, but all other PCC were statistically significant at p<0.05. The calculated gap closure at 60 h was 29±2% for PCX-0, 39±7% for PCC-1p, 50±8% for PCC-100p, 47±7% for PCC-10n, and 41±6% for PCX-10p. Immunofluorescent staining of samples previously used in the proliferation study revealed a higher extracellular deposition of laminin-5 (red fluorescence) in the PCC-100p group than in the PCX-0 group (FIG. 8C). Notably, HEKs grown on PCC-100p NFs were also generally larger and more elongated than those on PCX-0 NFs (FIG. 7C, FIG. 8C). These findings suggest a migratory phenotype consistent with the bioactivity of PCC-100p NFs shown in FIGS. 8A-8B.

### Discussions

Cells are known to be sensitive to stimulation by topography of their residing microenvironment.³⁶ Therefore, possible topographical effects (such as through differing fiber morphologies) needed to be ruled out for the cell studies of CRT effects (presence vs absence) herein. PCX-0 and PCC-100n NFs were comparable in terms of fiber uniformity as well as fiber diameter. As CRT is negatively charged, 37 the degenerated fiber morphology seen in the PCC-1 μ(FIG. 1A) NF group is likely due to the high CRT content altering the conductivity of the polymer solution beyond a certain threshold, thereby changing the electrospinnability.^{38,39} Even at this high CRT concentration, NFs comparable to the PCX-0 and PCC-100n groups could still be obtained, for example, by increasing the polymer concentration as previously shown.⁴⁰ Since fibroblasts were previously demonstrated to be sensitive to CRT exogenously at ng/ml levels^{9,10} and keratinocytes at pg/mL levels,⁹ the need for a higher concentration of CRT within the NFs did not appear to be warranted. Therefore, due to the apparent morphological similarities. PCX NFs and PCC NFs containing CRT at PCC-100n concentrations and below (outlined in Table 2) were used in these studies.

Concentration gradient was predicted to be the main driving force affecting the release of CRT from NFs through simple diffusion, considering PCL, the main component of the NFs, is a slow-eroding, hydrophobic polymer (FIG. 2B).⁴¹ Accordingly, in the prolonged release experiment, the full receiver volume was replenished with fresh PBS every 2-5 days to mimic cell culture media changes. This is consistent with the in vivo situation wherein the CRT concentration gradient would be reduced by diffusion out of immediate wound area, adsorption to surrounding ECM, and/or uptake by cells. In this way, release of CRT-FITC from PCCf-100n NFs was confirmed to be concentration-dependent, since the complete replenishment of the receiver volume resulted in a short burst release period of about 4 h at each round (even when the previous round had leveled off release), followed by a period of more steady release over the course of 2-5 days. Notably, the burst release is a result of the simple blend method used in this proof-of-concept study and could be reduced (i.e., release kinetics altered) in future studies by various means, as reviewed by Chou et al..⁴² Fluorescent imaging conducted throughout the release experiment corroborated the finding that CRT-FITC release was to a certain degree sustained over the course of at least 6 weeks, since green fluorescence attributed to CRT-FITC was still seen throughout the PCCf-100n NFs after 8 weeks submerged in PBS.

The wound environment contains abundant enzymes released by cells that are important in the process of healing a wound as well as commensal and pathogenic bacteria. While proteases are key players in wound debridement necessary for normal healing,⁴³ the abundant presence of proteases is a notable feature of chronic wounds, greatly contributing to their tissue damage and chronicity.⁴⁴ As such, the chronic wound proteolytic environment can degrade and inactivate therapeutic biological molecules rendering their topical application ineffective. One of the goals of this study was to demonstrate whether CRT incorporated into NFs by means of electrospinning would protect the molecule from proteolytic degradation. Fluorescence intensity of CRT-FITC in PBS contributed by PCCf-100n NFs compared to free CRT-FITC was used as an estimate of CRT stability afforded by NF protection (FIG. 3A-E). Notably, CRT appears not to be degraded by cathepsin G or elastase (two abundant enzymes in wound fluid), since fluorescence intensity remained unchanged for free CRT-FITC (i.e., even without NFs). Importantly, the results suggest that in comparison to free CRT-FITC in solution, CRT in NFs (PCCf-100n) was protected from proteolysis by subtilisin, a bacterial enzyme, and proteinase K, a potent substrate-specific enzyme. Nonetheless, CRT post release from NFs does not appear to be vulnerable to proteolytic digestion by elastase or cathepsin G since fluorescence was not increased with CRT in solution in PBSs. Whether the increase in fluorescence intensity upon incubation with these enzymes reflects the generation of proteolytic fragments or a conformational change in CRT exposing FITC-labeled amines was not determined.

A highly cellular granulation tissue with abundant collagen and other ECM proteins is critical for reconstruction of the wound defect. Fibroblasts are the most prominent cells of the dermis that migrate into the wound, proliferate and therein produce extracellular matrix proteins to compose the granulation tissue, which provides a substrate for keratinocytes to migrate from the wound edge for reepithelialization. Previous studies have demonstrated that CRT functions as a chemoattractant to recruit fibroblasts to reconstruct the wound, stimulate proliferation, upregulate α-smooth muscle actin (for wound contraction) and induce expression of integrins and induces ECM proteins.⁶¹ Importantly, CRT incorporated into NFs retained its biological activities of inducing proliferation (FIGS. 4A-4C), migration (FIGS. 5A-5C), and ECM protein and integrin expression (FIG. 6) by HFFs. The increased mitogenic response of HFFs cultured on PCC-100n (CRT electrospun into NFs) and PCX-100n (PCX plus 100 ng/ml exogenous CRT) was similar and statistically significantly higher on day 2 compared to HFFs grown on PCX-0 (no CRT). Thus, the increased proliferation observed in the PCC-100n and PCX-100n groups on day 2 indicates that the mitogenic activity of CRT was retained during its processing into NFs. Further, the CRT released from the NFs as conditioned media (CM), stimulated proliferation of HFFs grown on tissue culture plates (TCP), in a comparable manner to the intact (unprocessed) CRT (FIG. 4C). While CRT induced a statistically significant proliferative response in these NF experiments, the induction of HFF migration on NFs by CRT as measured by gap closure rate was not statistically significant. Significance appeared not to be attained because the PCX-0 group showed a high standard deviation, which might be related to random motility of individual cells on NFs. In contrast, HFFs cultured on PCC-100n NFs showed little variation in the motility assay (FIG. 5C) and the cells demonstrated directed migration (not random). Despite the lack of statistical significance, there was nonetheless a trend of CRT increasing the rate of gap closure observed at timepoints after 18 h, which was of borderline significance in the PCC-In group. As the peak response of HFF migration in a scratch assay on TCP was previously reported to occur at 1 ng/ml CRT with decreased activity obtained at higher concentrations,⁹ it is possible that borderline significance was only found with the PCC-In NFs in this experiment, since higher CRT concentrations might diminish the migratory response.

Nonetheless and importantly, the migration experiments shown herein were performed in the presence of mitomycin C to block proliferation. Clearly, the gap closure of HFFs on CRT NFs was wholly related to motogenicity (FIG. 10) since in the presence of mitomycin C, CRT induced migration whereas the migration in response to fetal bovine serum (contains many growth factors) indicated that proliferation played a major role in closing the gap in this migration assay. Many cell adhesion complexes have been shown to elicit mechanosensing effects, and vinculin is a particularly well characterized focal adhesion protein that links transmembrane adhesion receptors such as integrins to cytoskeletal components such as F-actin fibers to form adhesion complexes.⁴⁷ Whereas there was no apparent distinction between induction of migration by gap closure of HFFs by CRT on NFs, related to adhesion and migration, more prevalent vinculin staining was observed in HFFs cultured on PCC-100n NFs than on PCX-0 NFs, suggesting the formation of more mature cell adhesion complexes at cell-NF contacts. Previous work has uncovered a relationship between vinculin (i.e., focal adhesion complex) size and cell migration speed,⁴⁸ suggesting that HFFs on PCC-100n NFs may be more motile, which agrees with the observations in FIG. 5C, shown by time lapse photography. In addition to vinculin, the F-actin staining stretched across the cells suggests a more motile phenotype, and the stretched morphology with lamellipodia and filopodia observed in FIG. 6A and FIG. 6C evidently support cell motogenicity, as widely established.⁴⁹ Similar to vinculin, focal adhesion kinase (FAK), a cytoplasmic protein tyrosine kinase, is associated with mechanosignaling.⁴⁶ Notably, the increased lamellipodia protrusion, which is associated with Rac-dependent functions, and the pFAK staining observed for HFFs on PCC-100n NFs are consistent with cell adhesion and the migratory phenotype. Interestingly, another unique observation of HFFs cultured on PCC-100n NFs was the trail of pFAK-positive extracellular vesicles released by the cells. Based on the size, these vesicles implicate a migration-specific exosome recently identified as migrasomes.⁵¹

Previously, CRT was shown to induce ECM proteins and TGF-β1 by fibroblasts in vitro.¹² Further, CRT could induce ECM proteins via TGF-β1 Smad2/3 signaling, which was modulated by CRT ostensibly to ameliorate scarring.⁵² In addition, both α5 and β1 integrins (FIG. 6D, FIG. 9A) were induced by CRT. The studies herein demonstrate that HFFs seeded on CRT-NFs showed a comparable increase in integrin α5 (FIG. 11A) and integrin β1 in response to CRT for both NFs electrospun with CRT and NFs with CRT added exogenously. Therefore, in addition to the ability of CRT to promote cell motility and proliferation upon incorporation into NFs, the induction of integrin α5 and integrin β1, that together form an important receptor for cell migration on fibronectin,^{53.} which is also an important component of granulation tissue and collagen organization,⁵⁴ were also retained, consistent with known CRT bioactivities. It is interesting to note that high levels of TGF-β1 were detected on HFFs cultured on PCC-100n NFs and, since CRT modulates TGF-β1 to favor tissue regeneration, this suggests that CRT in PCC-100n NFs could have the same effect in vivo. The increase in TGF-β1 synthesized by HFFs in these studies appears to be unique and a synergistic activity of CRT electrospun into NFs versus exogenously added CRT to NFs (FIG. 6E, FIG. 11C).

Similar to previous findings in both in vitro and in vivo porcine and diabetic mouse wound experiments, CRT electrospun into NFs was able to retain its inductive functions to promote proliferative and migratory responses of human keratinocytes (HEK) (FIG. 7A-C, FIG. 8A-C). Distinct from the results obtained with HFFs, in which CRT electrospun into NFs (PCC-100n) or added exogenously to NFs (PCX-100n) exhibited similar support to cell proliferation, there was statistically significant increase in HEK proliferation only on CRT-NFs and not when cells were exposed to exogenously added CRT (FIG. 7A). A possible explanation of such might be based on the timing of the release of CRT from the NFs, in comparison to adding exogenous CRT. As such, HEKs are exposed to CRT within the NFs immediately upon seeding in the PCC-100p group, rather than adhesion to NFs prior to exogenously supplying CRT to the cells on NFs in the PCX-100p group. Accordingly, certain cellular effects may be elicited even prior to their attachment by the burst-released CRT of the CRT-NFs. Alternatively, exposure of CRT electrospun into the NFs is on the basal surface of the cell. which might elicit different signaling mechanisms allowing for the different proliferative response shown herein (FIG. 7A). Regardless, subsequent immunostaining with Ki67 of the HEKs supported the differences obtained between NFs with and without CRT. With respect to induction of HEK migration by CRT-NFs, while HEKs showed a greater response to CRT electrospun into NFs (e.g, PCC-100p vs. PCX-0), exogenously added CRT to HEKs on NFs (PCX-10p: used as 10 pg/mL exogenous control) also supported a migratory response of these cells. Moreover, consistent with migration, an increase in laminin-5 was observed in extracellular deposits near the migrating cells.

Taken together, the Examples presented above demonstrated that CRT-containing PCL/Col1-based electrospun NFs can be used as a biomimetic ECM. This hybrid scaffold was shown to (1) retain the biological activity of CRT while sustaining its release and protecting it against degradation by enzymes in a chronic wound bed and (2) provide synergism of biological and mechanical cues to further promote wound healing functions of cells.

### Novel Synergistic Effects of CRT Electrospun With NFs

The PCL/Col1 NFs reported herein incorporate CRT into biomimetic extracellular matrix (ECM) NFs for superior preservation of CRTs biological activities while achieving sustained release to exploit CRTs chemoattraction activity for tissue-healing cells onto (i.e., keratinocytes to resurface the wound) and into (i.e., fibroblasts into the wound to produce ECM proteins to reconstruct the wound defect) the NFs for injury repair. As such, the cells that are recruited into the wound would be expected to continuously respond to the biological effects of CRT and the novel synergistic effects of the hybrid CRT-NFs for sustained activity of promoting the wound healing process.

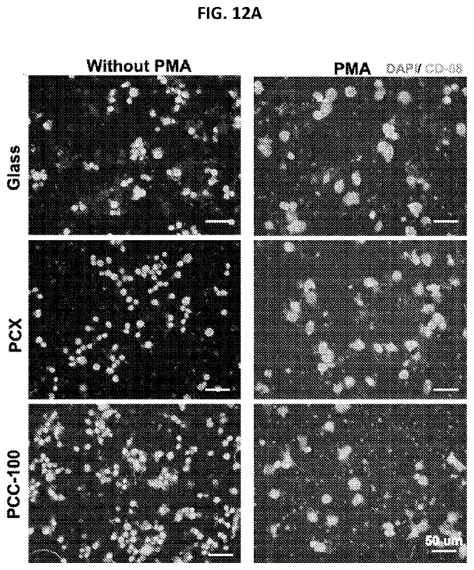

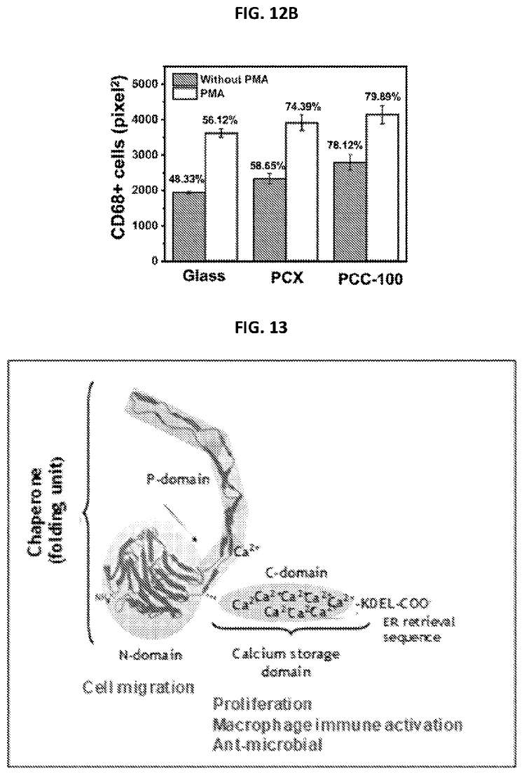

In one set of experiments, CRT sequestered into NFs by electrospinning retained the ability to induce proliferation and migration of human keratinocytes and fibroblasts with the same specific activity (potency) as previously shown for exogenous CRT directly added to these cells (FIGS. 4A-4C, FIGS. 5A-5C, FIGS. 7A-7C, FIGS. 8A-8C). In addition, monocytes added to CRT-NFs (PCC-100n) showed increased expression of CD68, cell surface cluster protein that is upregulated following activation of monocytes. In contrast, monocytes grown in the presence of NFs without CRT (PCX) were not activated (78% compared to 48%, FIGS. 12A and 12B).

Further, HFFs grown on PCC-100n retained the ability to induce TGF-1 (FIG. 11C), fibronectin (FIG. 6E), collagen (FIG. 11D), integrin α5 (FIG. 11A) and integrin β1 (FIG. 11B) important for migration on fibronectin, as shown at 2 days post-seeding (FIGS. 5A-5C), which was greater than PCX-0. In addition, the PCC-100n appeared to induce a higher level of TGF-β1 (key inducer of ECM proteins) compared to PCX-100n or PCX-0, suggesting a synergistic effect of CRT in NFs compared exogenously added CRT to PCX-0.

Compared to NFs without CRT, human fibroblasts (HFFs) seeded onto CRT-NFs demonstrated a more elongated migratory cell phenotype with extended lamellipodia and filopodia (FIGS. 6A-6F). As shown in FIGS. 6A-6F, pFAK (red fluorescence) was predominantly distributed in the perinuclear region of HFFs cultured on PCX-0 NFs, whereas more pFAK was located at the cell peripheries and even in extracellular vesicles left behind by the cells (FIG. 6A) on PCC-100n NFs. In addition, there was a marked increase in pFAK positive exosomes per area (FIG. 6A, referred to as migrasomes; both large and small vesicles) on the cell substratum of the PCC-100n compared to PCX-0 (FIG. 6G). The migrasomes appeared to be trailing the HFFs as migration ensued. Importantly, as shown, the synergy of CRT (electrospun into) NFs promoted a different more migratory phenotype of fibroblasts than NFs without CRT or exogenous CRT added to NFs.

In addition, a higher density of actin filaments that stretched across keratinocytes seeded onto CRT-NFs was observed compared to NFs without CRT, PCX-0 (FIG. 8C). In addition, based on immunostaining for laminin 5 (also known as laminin 332), more laminin 5 was deposited around the keratinocytes cultured on PCC-100n, CRT-NFs compared to NFs without CRT (FIG. 8C). As another example of synergistic activity of CRT electrospun into NFs (PCC-100n), keratinocytes were stimulated to proliferate on CRT electrospun into NFs (PCC0100p) but not on NFs with exogenously added CRT (PCX-100p) shown on day 3 after seeding (FIG. 7A). Furthermore, monocytes were activated to express CD68, a differentiation marker for macrophages, when added to CRT-NFs compared to NFs alone (FIGS. 12A and 12B).

Results reported herein show that PCX-0 NFs (without CRT) were a statistically significant poorer substrate for keratinocyte migration than PCC-100p, once again showing the synergism between CRT electrospun into PCL/Col1 NFs. This is surprising and unexpected, based on the observation that in past studies, keratinocytes showed preference to gelatin-coated PCL-Col NFs to promote and support migration of these cells.²⁴ Therefore, the CRT electrospun into the NFs appeared to compensate for a NF type that previously only weakly supported migration of HEKs.

In other aspects, the CRT-NFs reported herein provide biochemical and biological cues that recruit relevant cells into and over (for wound resurfacing, production of neodermis, and wound closure) the wound bed, induce cell proliferation to increase cell numbers that function in tissue repair, and promote the synthesis and release of new ECM proteins. These proteins form the granulation tissue required for the repair, reconstruction, and remodeling of all types of acute cutaneous wounds and “hard-to-heal” chronic wounds.

Chronic wounds are defined as remaining open for more than 8 weeks. Cell signaling for CRT functions is at least in part, via the LRP1 receptor. Cellular protein levels induced by CRT include, but are not limited to, collagens, fibronectin, laminins, elastin, TGF-β1, TGF-β3, p-FAK, Rho GTPase proteins, a-smooth muscle actin, and Wnt proteins. ^{12,63,66-68} Cellular processes shown to be upregulated by CRT-NFs include, but are not limited to, cell adhesion and related migration, calcium regulation (i.e., store operated calcium entry [SOCE]), cell differentiation, cytokine release, and phagocytosis of dead cells/debris and bacteria.^{52.69-75} Furthermore, CRT is an opsonin for gram-negative and gram-positive bacteria with potential beneficial anti-microbial effects.⁶¹ This is particularly important for chronic wounds with a notoriously high risk of infection. As shown by results reported herein, neither CRT nor NFs alone can achieve such potent functions (e.g., promotion of tissue regeneration). CRT-NFs are a unique and superior bioactive device because both the synergistic effects of CRT laden NFs and, the CRT-specific effects, are not inherent in NF ECM scaffolds. Since all functions of CRT tested in these experiments were retained by CRT electrospun into NFs, it is highly likely that other biological functions of CRT important to wound healing, not tested here would be retained by CRT sequestered in the NFs.

In vivo, in porcine (only mammal that heals similar to humans) and diabetic murine wound healing models, topically applied CRT induced proliferation of the basal and suprabasal keratinocytes, rapidly re-epithelializing the wounds with all four layers of epidermis compared to Regranex (PDGF-BB), which did not cause wound re-epithelization.^{9,52} In addition, pM concentrations of CRT induce ECM proteins by fibroblasts, in vitro, which is important for the treatment of deep tissue injuries to reconstruct and remodel in the deep wound gap. ECM/granulation tissue formation, in vivo, is exemplified by the appearance of a highly cellular and collagen-laden neodermis shown in the wounds topically treated with CRT in saline [for 4 days] compared to the controls with only saline treatment.^{9,10} Uniquely, CRT shows tissue regenerative effects, namely epidermal appendage (i.e., hair follicles and sebaceous glands) neogenesis [of stem cells] and lack of scarring.¹⁰ Tissue regenerative qualities have not been shown by NFs alone or any other protein, other than CRT. Teleologically, CRT imparts tissue regenerative functions to NFs. The biological functions of CRT can be prolonged and thus, more effective, by incorporating CRT in a matrix, such as NFs, for sustained release (FIGS. 2B-2C, FIG. 1D). Together with tunable NFs, CRT release can be regulated and the synergistic effects of CRT, as the biological cues, and NFs, harboring an array of instructive mechanical cues, are presented in CRT-NFs for a precision medicine approach to tissue regeneration.

Unusual and surprising attributes of the embodiments herein are that CRT biological functions important to wound healing are retained following the electrospinning procedure along with unaltered NF dimensions (FIGS. 1A and 1B, Table 1). In addition, CRT is stable in an organic solvent, such as hexaflouro isopropanol (HFIP) shown here. and during exposure to the high voltage electric field used throughout the fabrication procedure to enable the production of the hybrid NFs containing CRT. Specifically, CRT retains its full molecular mass of 46,460 Daltons (is not fragmented) and is properly folded such that its 3-dimensional structure retains full potency for all wound repair-related biological activities tested. Certain proteins have been electrospun into NFs composed of different nanofiber chemical material. For example, Wang et al has electrospun TGF-β1 into NFs and showed that it induces myoblast differentiation of fibroblasts.⁶² However, TGF-β1 is classically known to cause fibrosis and scarring during wound healing. Notably, CRT has been shown to modulate TGF-β1 activity and induce tissue regeneration in vivo in mice hallmarked by pigmented hair follicle and sweat gland neogenesis, and lack of scarring (shown at day 28 post-wounding).^{10,12} No other biological matter, other than CRT, has been shown to induce such “true” tissue regeneration.

The CRT-NFs reported herein also have the potential to foster and improve the rate and quality of wound healing. A priori, the NF physiological ECM as a wound treatment provides a matrix scaffold that precedes the requirement for cellular migration into wounds for subsequent ECM induction. Without granulation tissue (neo-dermis/ECM) produced by fibroblasts and migratory stem cells (fibrocytes) from the bone marrow, keratinocytes cannot migrate from the wound edges (epithelial tongues) to re-epithelialize the wound. Specifically, the ECM substrate deposited by fibroblasts is a provisional matrix for wound epithelialization and closure. Advantageously, the ECM provided by the NFs with CRT incorporated is thus already present for fibroblasts and keratinocytes to be recruited into the wound, to proliferate in response to CRT-NFs (PCC-100n) and therein, further induce and accumulate ECM proteins produced by fibroblasts such as collagens and fibronectin as the functional provisional matrix. A novelty of the presence of CRT-NFs (PCC-100n) in a wound ostensibly decreases the time for ECM induction resulting in more rapid progression to the remodeling phase of wound healing. In other words, the physiological scaffold is a preformed advantage for ECM production by fibroblasts and mesenchymal cells that have been recruited into the wound by CRT sequestered in the NFs.

In another aspect, the CRT-NFs reported herein were shown to subvert the need for multiple in vivo applications of CRT to wounds. Traditionally, CRT is topically applied for multiple (e.g., 4) consecutive days in porcine and diabetic murine wound healing/tissue regeneration experiments.^{9,10} Following the application of CRT-NFs to the wound, 77% of the CRT electrospun into NFs was released over 3 days with continued release for up to 8 weeks (FIGS. 2B and 2C, FIG. 1D). This represents an improvement in the current method for topical use of CRT in saline which was applied every day for 4 days. Moreover, a benefit of PCL/Col1 NFs or NFs composed of other materials is that the fibers can be modified and/or crosslinked to achieve slower release if this is beneficial and superior for CRT-NFs in wound healing rather than CRT in saline.

CRT electrospun into NFs interacts with, and putatively stimulates, receptors on the basal side of cells that are adherent to the CRT-NFs. In addition, CRT is released from the NFs (FIGS. 2B and 2C, FIG. 1D), which binds to cell receptors on the apical side of the cells. Without CRT electrospun into NFs, there would not be cell basal side exposure for unique cell stimulated functions. Additionally, activation of the cytoskeleton (actin and myosin) and proteins involved in lamellipodia and filopodia formation were detected, indicative of the migratory phenotype (actin filament formation, vinculin [adhesion activation], rac1 [family of Rho GTPases]) in keratinocytes and fibroblasts seeded on CRT-NFs. The differential activation of cell receptors on the basal and apical parts of the cell is novel, which as shown in this invention, elicits different cellular functions and dictates specific cell morphology (FIGS. 8A-8C).

Proteases are important in the inflammatory phase of wound healing stage of wound healing. Growth factors and other proteins important to healing wounds are subjected to proteolytic digestion by the high enzymatic activity in the wound bed that is derived from neutrophils and bacteria and other cells that have migrated into the wound bed. Results reported herein show that CRT in NFs is protected from digestion by physiologically relevant wound enzymes compared CRT unprotected in a tube without NFs.

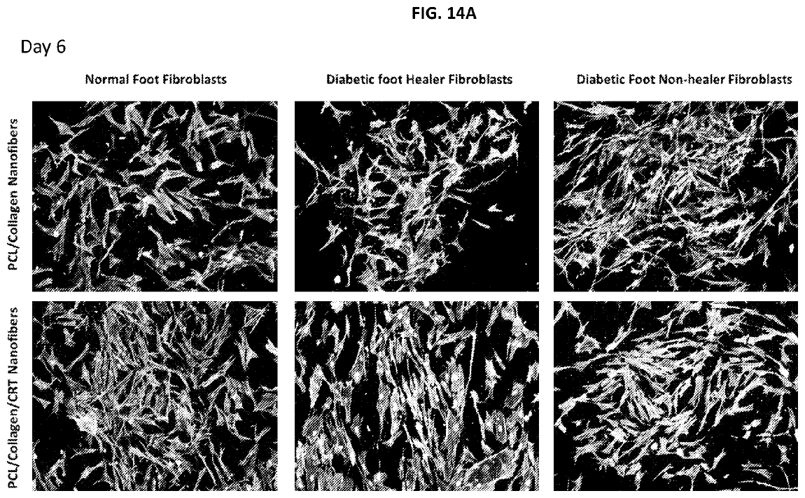

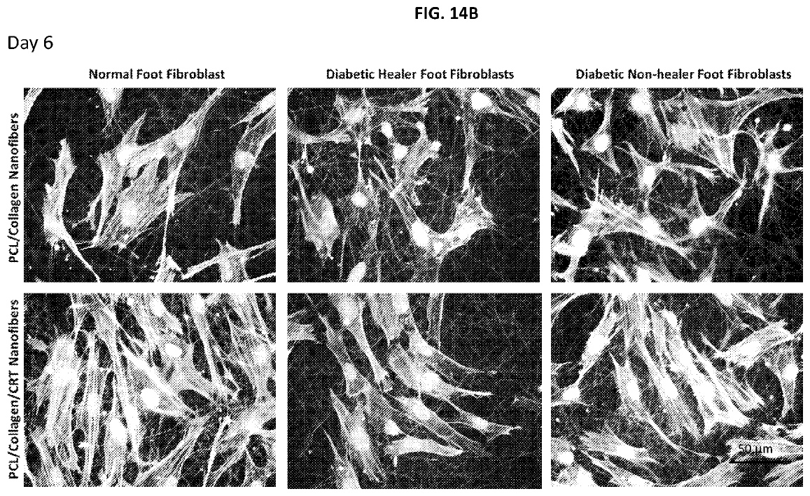

FIG. 13 shows a map of the specific/separate biological functions of CRT associated with wound healing to the domains. These functions were shown by producing CRT domains and fragments recombinantly by bacteria (E. coli) and employing the domains in wound healing specific bioassays. The domains retained the same specific activity (potency) as the intact CRT molecule. Electrospinning CRT N, P, and/or C domains into NFs has the potential to provide a precision medicine approach to individual patient wound healing/care. For example, the CRT N Domain elicits cellular migration while the P domain induces the synthesis of ECM proteins. A higher ratio of N domain or P domain compared to the intact CRT would, for example, provide healing for large surface area wounds, such as burns, and deep tissue wounds, such as deep severe injury, respectively.

In summary, CRT electrospun into nanofibers not only retains calreticulin's beneficial wound healing effects but synergizes with the physiological PCL/Col11 ECM-like fiber scaffolds for improved and newly discovered different synergistic activities related to tissue regeneration.

### Example 6. Calreticulin Electrospun Into Nanofibers (CRT-NFs) Induced a Motogenic (migratory) Cell Phenotype of Diabetic Fibroblasts Isolated From Non-healing Diabetic Foot Ulcers/wounds

Fibroblasts, isolated from diabetic foot ulcers that have healed within 12 weeks after becoming wounds by injury or other means and those that have not healed before 12 weeks, have been recently characterized and compared by single cell RNA sequencing (Theocharidis, G. et al., 2022, Nature Communications, 13:181, 27801-8). In addition, in this study, both phenotypes (M1 and M2) of macrophages were compared by immunofluorescent staining using specific antibodies on plantar foot tissue preparations, and peripheral blood mononuclear cells (PBMCs) were compared from diabetic healers and non-healers. Fibroblasts from diabetic foot ulcer wounds that healed expressed higher levels of Metaloproteinase-1 (MMP1), MMP3, MMP11, HIF1A (hypoxia-inducing factor), CHI3L1, and TNFAiP6 than the fibroblasts from non-healing wounds. From these data, it was concluded that diabetic wounds that healed were more central in the wound bed, adopted a more acute inflammatory state consistent with M1 macrophages, and were involved in active wound remodeling to fully heal a DFU.

The previously characterized healer and non-healer fibroblasts with associated background data from macrophages and PBMCs were seeded and cultured at 37° C., 5% CO₂. Nanofibers (NFs) were prepared as described in Example 1 containing 100 ng/ml CALR electrospun into the nanofibers (CALR-NFs; PCC100n) and NFs without CALR (PCX-0) were prepared. The electrospun NFs were collected on glass coverslips that were coated with BPEI and placed in each well of a 6-well plate (Stack et al 2022). Normal foot fibroblasts (from a patient without diabetes, NFF14), healer fibroblasts (DFU7), and non-healer fibroblasts (DFU6) were seeded onto coverslips or onto the PCC100n or PCX-0 NFs on top of the coverslips. After 8 hours, CALR was added only to the cells seeded on the coverslips to a final concentration of 100 ng/ml.

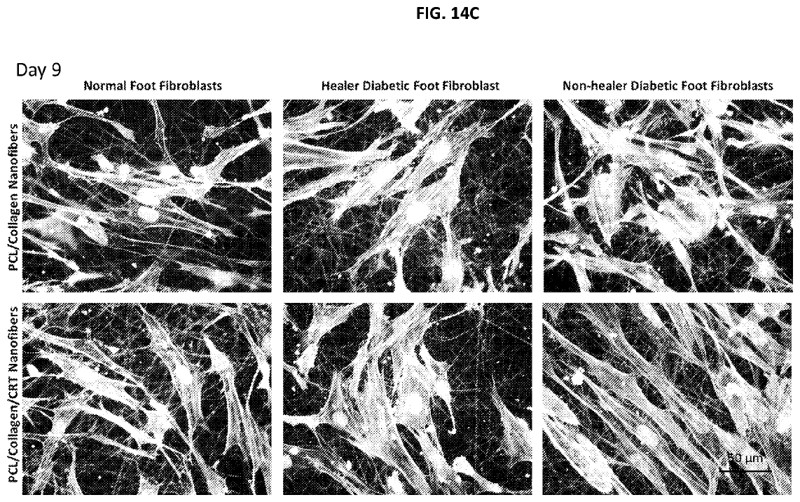

The NFF14 (normal plantar foot fibroblasts), DFU7 (healer plantar foot fibroblasts), and DFU6 (non-healer plantar foot fibroblasts) were treated with 100 ng/ml CALR on glass slides, the PCC100n (CALR electrospun nanofibers, CRT-NFs), and NFs alone (PCX-0) were fluorescently stained for F-actin with phalloidin at 8 hours, 6 days, and 9 days post seeding of the cells. The cells on glass coverslips and on NFs were observed by confocal microscopy (Zeiss 880) and images were captured at 10× and 40× (FIGS. 14A-14C).

Importantly, the non-healer and healer fibroblasts had an identical morphology on CALR-NFs suggesting that CALR-laden NFs have the potential to instruct non-healing DFU fibroblasts to adapt the phenotype of DFU healer fibroblasts. As the morphology of the cells grown on CALR-NFs is reminiscent of motogenic/migratory fibroblasts and inherent defects in cellular migration characterize poorly healing DFUs, CALR-NFs appear to be able to correct this medical problem as a therapeutic intervention of poorly or non-healing chronic wounds, a manifestation of various systemic diseases.

### Example 7. Calreticulin-electrospun Nanofibers (CALR-NFs) Stimulate Proliferation of Fibroblasts Isolated From Healer and Non-healer Diabetic Foot Ulcers (DFUs) Compared to Nanofibers Without CALR, Thereby Correcting an Inherent Defect in Non-healer Fibroblasts Associated With Non-healing Diabetic Foot Ulcers (DFUs)

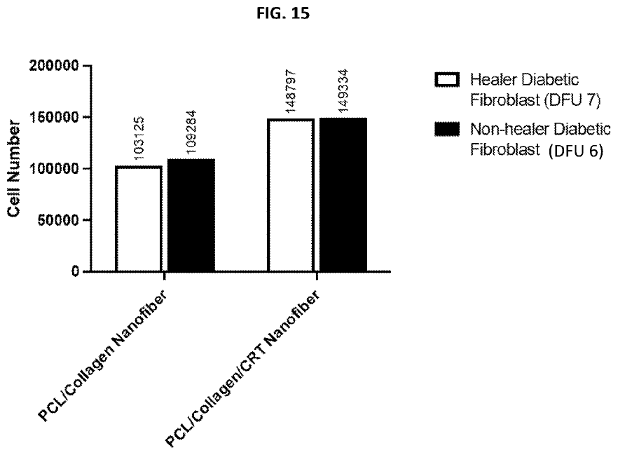

To quantify and compare proliferation of healer and non-healer fibroblasts on the CALR-electrospun nanofibers (PCC100n) vs. the PCL-Col1 nanofibers alone (PCX-0), the cells were seeded and cultured as described above and cell proliferation was determined by counting the number of cells on the entire nanofiber field. After 6 days in culture, the cells were fixed as described above and stained with DAPI. Images at 4× were captured using a BioTek Cytation C10 microscope. 8×8 captured images of 64 fields were stitched together and the cell number was determined by using BioTek Gen5 software. Both the non-healer (DFU6) and healer (DFU7) fibroblasts demonstrated an equal number of cells grown on NFs alone and CALR-NFs (FIG. 15). Importantly, the data show that CALR-NFs induced a 30% increase in the number of cells for both the healer and non-healer fibroblasts. As diabetic fibroblasts are defective in proliferation, the data show that CALR-NFs have the potential to correct this defect and promote normal wound healing of DFUs

### Materials & Methods

Below are the materials and methods used in the Examples described above.

### Materials

Recombinant human CRT expressed in Escherichia coli was obtained from Intas Pharmaceuticals Ltd. (Ahmedabad, India) and used from a 6.4 mg/mL stock solution in Tris-Ca buffer (TC; 10 mM Tris, 3 mM CaCl₂, pH 7.0). Human calreticulin isolated by a modification of Ciplys et al was used for all the studies described herein. For selected experiments, CRT was fluorescently tagged with fluorescein isothiocyanate isomer I (FITC, from Sigma-Aldrich) under basic conditions to obtain CRT-FITC.⁶⁰ Glass coverslips and calcium chloride solution (1.0 M) were obtained from Carolina Biological Supply Company. Tris buffer solution (2 M) and sodium carbonate were purchased from Acros Organics. Collagen type I (Col1, lyophilized from calf skin) was acquired from Elastin Products Company. 1,1,1,3,3,3-hexafluoro-2-propanol (HFIP) was obtained from Oakwood Products. Polycaprolactone (PCL, average Mn 80 kDa), branched polyethylenimine (BPEI, average Mw 750 kDa), mitomycin C (10 mg/mL in dimethyl sulfoxide (DMSO)), and sodium bicarbonate were obtained from Sigma-Aldrich. Elastase from human neutrophils, cathepsin G from human neutrophils, proteinase K from Tritirachium album, and subtilisin A from Bacillus licheniformis were purchased from Millipore Sigma and reconstituted as per manufacturer's protocol. All cell culture media and additives were from Gibco with the exception of keratinocyte basal media (KBM, Lonza), L-glutamine (Corning), fetal bovine serum (FBS, Atlanta Biologicals), and trypsin-EDTA for primary cells and trypsin neutralizing solution from American Type Culture Collection (ATCC).

### Fabrication and Characterization of CRT-containing Fibrous Matrices

Glass coverslips were cleaned with isopropanol then coated with BPEI (0.2 mg/mL, pH 9) for 20 min and blotted dry to improve subsequent adherence of electrospun fibers. NF matrices were prepared using an electrospinning technique.^{28,29} Briefly, PCL and Col1 (3:1 w/w) dissolved in HFIP overnight at 4° C. were mixed with CRT/TC solution at a volume ratio of 7:3 for 1 h at 4° C. under stirring (final polymer concentration of 10% w/v PCL/solvent). The final concentrations of CRT in various polyblend electrospinning solutions are shown in Table 2. For the study of CRT release kinetics and stability in the presence of enzymes, CRT-FITC was similarly electrospun into NFs (PCCf-100n). Upon loading into a 1-mL syringe with a 21 G stainless steel blunt-tip needle, the electrospinning solution was dispensed at a flow rate of 0.6 mL/h through an electric field of 1 kV/cm to fabricate NFs, which were collected onto the BPEI-coated glass coverslips on top of grounded aluminum foil. A 4-min collection time was chosen to assure sufficient NF coverage of the glass coverslips. Ambient conditions during electrospinning were 10-58% relative humidity and 20.5-27.9° C. The morphology of various NF matrices was examined by scanning electron microscopy (SEM; Zeiss Auriga Small Dual-Beam Field Emission) with a voltage of 1 kV and a working distance of approximately 5 mm. Fiber diameters were determined by using ImageJ to measure 50 fibers across 5 randomly selected SEM fields. Localization of CRT-FITC within NF matrices was examined with an EVOS M7000 Imaging System (ThermoFisher Scientific).

TABLE 2
Design of CRT-containing PCL/Coll nanofiber (NF) matrices.

| | | |
|---|---|---|
| Designated | CRT concentration | Anticipated CRT |
| Abbreviation | in electrospinning | concentration |
| of NF matrices | solution | releasable from NFs |

| | | |
|---|---|---|
| PCX | None | None |

| | | | | |
|---|---|---|---|---|
| PCC-1μ | 900 | μg/mL | 1 | μg/mL |
| PCC-100n | 90 | μg/mL | 100 | ng/mL |
| PCC-10n | 9 | μg/mL | 10 | ng/mL |
| PCC-1n | 900 | ng/mL | 1 | ng/mL |
| PCC-100p | 90 | ng/mL | 100 | pg/mL |
| PCC-1p | 900 | pg/mL | 1 | pg/mL |

CRT, calreticulin;
PCL, polycaprolactone;
Col1, type I collagen;
PCX, PCL/Col1;
PCC, PCL/Col1/CRT

### CRT Release From CRT-containing NFs

Release experiments were performed with 15-mm diameter PCX and PCCf-100 NF samples inside a 24-well plate with receiver volumes of 700 μL phosphate buffered saline (PBS) each. Plates were sealed with a plate sealer and wrapped in parafilm and aluminum foil to prevent evaporation and light exposure followed by incubation at 37° C. At designated timepoints, a 70 μL sample (i.e., 10%) was removed from each well and replaced with fresh PBS to maintain a constant receiver volume. The fluorescence intensity of these samples was immediately recorded with a Biotek Synergy H1 Microplate Reader through a green filter and used to calculate the cumulative mass of CRT-FITC released from the NFs against a standard curve. For prolonged release studies, the receiver volume was completely replenished (i.e., 100%) with fresh PBS every 2-5 days and the NF matrices were also imaged immediately after submerging in fresh PBS with an EVOS M7000 Imaging System (ThermoFisher Scientific).

### Proteolytic Resistance of CRT-containing NFs

To examine the susceptibility of CRT to proteolytic degradation following electrospinning into NFs, CRT-FITC (free) or PCCf-100n (CRT-FITC incorporated into NFs) were subjected to treatment with elastase, subtilisin, cathepsin G, and proteinase K separately. CRT-FITC/PBS solution (3.1 nM) or 15-mm diameter PCCf-100n samples submerged in PBS were incubated with elastase or cathepsin G at a molar ratio of 1:10 or with subtilisin or proteinase K at a weight ratio 1:10 or 1:100 (enzyme: CRT), and the reaction was carried out under static conditions at 37° C. Starting at 15 min and then every 20 min thereafter, a sample of the supernatant was collected followed by measuring its fluorescence intensity using a Biotek Synergy Neo2 Hybrid Multi-Mode Microplate Reader (excitation wavelength: 485 nm, emission wavelength: 528 nm). After 275 min, the reaction was terminated by addition of 1 mM phenylmethylsulfonyl fluoride. The data were expressed as fold change in fluorescence intensity of the sample (free CRT-FITC or PCCf-100n) with enzyme treatment at 275 min to fluorescence intensity of the same sample type without enzyme treatment at 15 min.

### Cell Culture

Primary human neonatal foreskin fibroblasts (HFF; CRL-2091, CCD-1070Sk, ATCC) were cultured in complete Minimum Essential Medium (MEM) containing 10% FBS, 2 mM L-glutamine, 1 mM sodium pyruvate, 50 IU/mL penicillin, and 50 μg/mL streptomycin with media was refreshed every 3-4 days. Cells were passaged at 60-70% confluency and used for experiments at passages 8-11. Telomerase-immortalized human keratinocytes (HEKs), a past gift from Dr. James Rheinwald (NIH Harvard Skin Disease Research Center), were cultured in complete Keratinocyte Serum-Free Medium (KSF) containing 50 μg/mL bovine pituitary extract (BPE), 5 ng/ml epidermal growth factor (EGF), and 0.3 mM additional CaCl₂, 100 IU/mL penicillin, and 100 μg/mL streptomycin with media refreshed every 2-3 days. Cells were passaged at 50-60% confluency. All cellular experiments on NFs were performed using 22×22 mm samples with the exception of the wound healing assay which was performed using 15 mm diameter samples. All NF samples were sterilized using ultraviolet irradiation (20 min per side) prior to use, and all treatment media contained 1% TC to equalize the effects of the exogenous CRT vehicle.

### Resazurin Assay for Cell Proliferation

To evaluate proliferation, cells were seeded onto NF matrices in a 6 well plate or directly onto tissue culture plastic (TCP) of a 96-well plate. For HFFs, cells were seeded at a density of 5.7 ×10³ cells/cm² and allowed to adhere for 6-8 h prior to further treatment. Complete MEM media with reduced FBS (i.e., 0.5%) was used to minimize the contribution of serum, except for the FBS positive control (i.e., PCX-FBS with 5% FBS). Conditioned media (CM) refers to media containing released CRT that was obtained by soaking PCC-100n NFs in FBS-reduced complete media for 1 day and respectively diluted 10-fold and 100-fold with fresh FBS-reduced complete media to obtain the estimated CRT concentrations of 10 ng/ml and 1 ng/ml. For HEKs, cells were seeded at a density of 1.0×10^{∝}cells/cm² and allowed to adhere overnight prior to further treatment. Complete KSF media was used throughout the experiment. For groups with exogeneous CRT treatment, cells were seeded on PCX NFs and then CRT was supplemented into the media to reach the designated concentration (e.g., 10 ng/ml for PCX-10n group). At predetermined timepoints, resazurin (Biotium) was mixed with either 0.5% FBS-complete MEM (for HFFs) or KBM (for HEKs) at a 1:9 volume ratio and then incubated with cells (1 mL/well). After an appropriate incubation period, 100 μL of supernatant from each well was transferred to a 96-well plate, and the absorbance at 570 nm and 600 nm was recorded using a BioTek Synergy H1 Microplate Reader. The net absorbance of each sample at each timepoint was normalized to the average value of the negative control at the initial timepoint in order to determine proliferation.

### Cell Motility Assay

To evaluate motogenic capacity, samples of NF matrices on glass coverslips were secured inside wells of a 6-well plate.⁶⁰ HFFs were seeded at a density of 5.7×10³ cells/cm² and allowed to adhere for 6 h prior to various treatments using 0.5% FBS-complete MEM. Immediately following treatment, samples were automatically imaged every 20 min for the next 12 h using a Biotek Cytation C10 Confocal Imaging Reader. Acquired images were analyzed in ImageJ. First, Boolean OR and subtraction operators were first used to attenuate the NF background and identify individual cells and the Manual Tracking plugin in ImageJ³⁰ was used to track 10 cells per condition.

### Wound-gap Closure Assay

To further evaluate motogenic capacity, a wound-gap closure analysis was performed with both HFFs and HEKs on NF matrices. Briefly, samples of NF matrices on glass coverslips were secured inside wells of a 24-well plate.⁶⁰ To better monitor the gap closure, cells were labeled with Vybrant™ DiD (Invitrogen) according to the manufacturer's instructions (15 min for HFFs, 10 min for HEKs). CytoSelect™ Wound Healing Assay inserts (Cell Biolabs, Inc.) were used to create a wound-gap of 0.9 mm on the surface of each NF sample; cells were seeded onto each side of the insert (4.5×10⁴ per side for HFFs, 1.0×10⁵ per side for HEKs) and allowed to adhere overnight. Immediately upon removal of the inserts, the cells were treated with mitomycin C (5 μg/mL, 1 h) to block proliferation (n.b., so that migration of cells observed would not be due to proliferation into the wound-gap). Then the cells were incubated in respective treatment media (0.5% FBS-complete MEM for HFFs, complete KSF without BPE and EGF for HEKs) and gap closure was monitored periodically by imaging with an EVOS M7000 Imaging System (ThermoFisher Scientific). Gap closure with DiD labeling was analyzed in ImageJ. First, red channel images of the full surface (stitched by the EVOS M7000 software) at different timepoints were aligned using the StackReg plugin available in ImageJ³¹ and cropped to the area of interest. Then, gap area was quantified using a macro based on a previous report,³² and the gap closure was quantified as a percent relative to the initial timepoint. In some experiments where DiD-labeling and mitomycin C pretreatment was not used per a previously published method,²⁹ the culture was fixed at the designated time and then the cells were stained with methylene blue (0.25 mg/mL, 15 min) and imaged using an EVOS M7000 Imaging System.

### Immunofluorescent Staining

Cultures were fixed with 4% paraformaldehyde for 10 min at room temperature and stored in PBS at 4° C. until use. Cells were then permeabilized with 0.1% Triton X-100 for 5 min and blocked for nonspecific binding with 3% w/v bovine serum albumin (BSA)/PBS for 5 min. All antibodies were diluted in 3% w/v BSA, and the primary or secondary antibody incubation was performed at room temperature for 1 h with gentle shaking. Primary antibodies used were as follows: rabbit anti-phosphorylated focal adhesion kinase (anti-pFAK; abcam, 1:400), mouse anti-keratin-1/10 (K1/10; Santa Cruz Biotechnology, 1:50), mouse anti-laminin-5 (EMD Millipore, 1:200), FITC-conjugated mouse anti-vinculin (Sigma-Aldrich, 1:100), FITC-conjugated mouse anti-Ki67 (Santa Cruz Biotechnology, 1:50), Texas Red-conjugated phalloidin (Biotium, 1:100), and Alexa Fluor™ 488-conjugated phalloidin (Invitrogen, 1:200). All conjugated secondary antibodies were from Jackson ImmunoResearch (1:50-1:100). Immediately following staining, samples were washed with PBS, mounted with DAPI, and sealed with clear nail polish. Imaging was performed using a Nikon Eclipse 80i epifluorescence microscope. The number of pFAK+ vesicles were semi-quantified using the CellProfiler image analysis software.³³ Briefly, green and blue channels were subtracted from the red channel images to reduce artifacts using the ImageMath module. Then, the number of pFAK+ vesicles in each image was counted using the IdentifyPrimaryObject module, with a pixel size threshold set between 3 and 60.

### Western Blot Analysis

To determine protein induction by CRT on NFs, Western blot analysis was employed. Briefly, HFFs were seeded onto NFs at a density of 5.7×10³ cells/cm² and allowed to attach for 6-8 h, treated with appropriate media (i.e., FBS-reduced complete media with or without 100 ng/ml CRT) for 48 h, and cell lysates were prepared with 1× RIPA lysis buffer (Sigma) containing 1× protease inhibitor cocktail on ice. The protein concentration of each sample was determined using a Micro-BCA protein assay kit, and 15 μg of each protein sample in Laemmli buffer containing 5% β-mercaptoethanol was loaded on SDS-PAGE (10% acrylamide) and transferred onto PVDF membrane for immunoblotting. The blots were blocked in 5% of nonfat dry milk in TBST for 1 h followed by overnight incubation in primary antibodies at 4° C.: mouse anti-human fibronectin (BD Biosciences, 1:1000) or mouse anti-integrin β1 (Santa Cruz Biotechnology, 1:500). To determine the levels of TGF-β1, the membrane was blocked overnight in 5% nonfat dry milk at 4° C. before incubating with the rabbit antibody³⁴ (2 μg/mL in 3% of nonfat milk in TBST) for 4 h at room temperature. β-actin (1:10000 in 5% nonfat dry milk) was used as a loading control in all experiments. After incubation with primary antibodies, the membranes were washed thrice with TBST followed by addition of secondary antibody for an incubation period of 1.5 h. goat anti-mouse (Invitrogen, 1:2000 in 5% nonfat dry milk in TBST) or goat anti-rabbit (Invitrogen, 1:2000 in 5% nonfat dry milk in TBST). The proteins transferred onto the membranes were detected using chemiluminescence-based SuperSignal West Femto Maximum Sensitivity Substrate (Invitrogen) and the image was captured using a ChemiDoc MP Imaging System (Bio-Rad). Densitometric scanning was performed using ImageJ, and the intensity of each band was normalized to β-actin. The data are expressed as fold change for each target protein in treatment groups over the PCX-0 control.

### Treatment of Fibroblasts With CALR/CRT and CALR/CRT-NFs

Healer and non-healer fibroblasts were cultured in Dulbeccos Minimum Essential Media (DMEM; high glucose), 1% HEPES buffer, 10% Fetal Bovine Serum (FBS), and Pen-Strep until 80% confluent. Polycaprolactone/collagen 1 (PCL/Col1) nanofibers (NFs) were prepared as described in Example 1 containing 100 ng/mL CALR electrospun into the nanofibers (CALR-NFS; PCC100n). Similarly, NFs without CALR (PCX-0) were prepared. The electrospun NFs were collected on (22×22 cm) BPEI-coated coverslips and placed in each well of a 6-well plate. Ten thousand cells (1.0×10⁴) of normal foot fibroblasts (from a patient without diabetes, NFF14), healer fibroblasts (DFU7), and non-healer fibroblasts (DFU6) in complete media were separately seeded onto coverslips or onto the PCC100n or PCX-0 NFs on top of the coverslips. The media was replenished every other day. For those cells cultured on the coverslips, after 8 h, CALR was added to the cell culture at a final concentration of 100 ng/ml.

The NFF14 (normal foot fibroblasts), DFU7 (healer fibroblasts), and DFU6 (non-healer fibroblasts) were incubated on the glass cover slips (treated with 100 ng/ml CALR), PCC100n (CALR electrospun nanofibers, CRT-NFs), and NFs alone (PCX-0) were fluorescently stained for f-actin with phalloidin at 8 hours, 6 days, and 9 days post seeding of the cells. For fluorescence staining, the cells were washed with PBS, fixed with 4% paraformaldehyde, permeabilized with 0.1% Triton X-100 for 5 minutes, and blocked with 3% (w/v) bovine serum albumin (BSA)/PBS for 5 minutes. Phalloidin conjugated with sulforhodamine-Texas Red (Biotum, Cat #00033) diluted at 1:200 in PBS was added and the cells were incubated for 30 minutes at room temperature. The cells on glass coverslips and on NFs were observed using a Nikon Eclipse 80i epifluorescent microscope and images were captured at 10× and 40×.

### Statistical Analysis

All values are shown as average±standard deviation. Statistical testing is described in the results section; p<0.05 was considered significant [p<0.05(*), p<0.01 (**), and p<0.0001(****)] and 0.05≤p<0.1 was considered borderline significant (#).

### References

(1) Brem, H.; Stojadinovic, O.; Diegelmann. R. F.; Entero, H.; Lee, B.; Pastar, I.; Golinko, M.; Rosenberg. H.; Tomic-Canic, M. Molecular Markers in Patients with Chronic Wounds to Guide Surgical Debridement. Mol. Med. 2007, 13 (1-2), 30-39. doi.org/10.2119/2006-00054.Brem.
(2) Armstrong, D. G.; Boulton, A. J. M.; Bus, S. A. Diabetic Foot Ulcers and Their Recurrence. N. Engl. J. Med. 2017, 376 (24), 2367-2375. doi.org/10.1056/NEJMra1615439.
(3) Boulton. A. J. M.; Vileikyte. L.; Ragnarson-Tennvall, G., Apelqvist, J. The Global Burden of Diabetic Foot Disease. Lancet Lond. Engl. 2005, 366 (9498), 1719-1724. doi.org/10.1016/S0140-6736 (05)67698-2.
(4) Jupiter. D. C.; Thorud, J. C.; Buckley, C. J.; Shibuya, N. The Impact of Foot Ulceration and Amputation on Mortality in Diabetic Patients. I: From Ulceration to Death, a Systematic Review. Int. Wound J. 2015, 13 (5), 892-903. doi.org/10.1111/iwj.12404.
(5) Margolis. D. J.; Bartus. C.; Hoffstad. O.; Malay. S.; Berlin. J. A. Effectiveness of Recombinant Human Platelet-Derived Growth Factor for the Treatment of Diabetic Neuropathic Foot Ulcers. Wound Repair Regen. 2005, 13 (6), 531-536. doi.org/10.1111/j.1524-475X.2005.00074.x.
(6) Walters. J.; Cazzell, S.; Pham, H.; Vayser. D.; Reyzelman, A. Healing Rates in a Multicenter Assessment of a Sterile, Room Temperature, Acellular Dermal Matrix Versus Conventional Care Wound Management and an Active Comparator in the Treatment of Full-Thickness Diabetic Foot Ulcers. Eplasty 2016, 16, e10.
(7) Marston. W. A.: Hanft. J.; Norwood. P.; Pollak, R.; Dermagraft Diabetic Foot Ulcer Study Group. The Efficacy and Safety of Dermagraft in Improving the Healing of Chronic Diabetic Foot Ulcers: Results of a Prospective Randomized Trial. Diabetes Care 2003, 26 (6), 1701-1705. doi.org/10.2337/diacare.26.6.1701.
(8) Driver, V. R.; Lavery. L. A.; Reyzelman, A. M.; Dutra, T. G.; Dove, C. R.; Kotsis, S. V.; Kim, H. M.; Chung, K. C. A Clinical Trial of Integra Template for Diabetic Foot Ulcer Treatment. Wound Repair Regen. 2015, 23 (6), 891-900. doi.org/10.1111/wrr.12357.
(9) Nanney. L. B.; Woodrell, C. D.; Greives, M. R.; Cardwell, N. L.; Pollins. A. C.; Bancroft. T. A.; Chesser. A.; Michalak, M; Rahman. M.; Siebert, J. W.: Gold, L. I. Calreticulin Enhances Porcine Wound Repair by Diverse Biological Effects. Am. J. Pathol. 2008, 173 (3), 610-630. doi.org/10.2353/ajpath.2008.071027.
(10) Greives. M. R.; Samra. F.; Pavlides, S. C.; Blechman. K. M.; Naylor, S. M.; Woodrell, C. D.; Cadacio, C.; Levine. J. P.; Bancroft, T. A.; Michalak, M.; Warren, S. M.; Gold, L. I. Exogenous Calreticulin Improves Diabetic Wound Healing. Wound Repair Regen. 2012, 20 (5), 715-730. doi.org/10.1111/j.1524-475X.2012.00822.x.
(11) Gold. L.; Pavlides. S.; Ojeda, J.; Eaton, B.; Panchal, R. A New Role for the Multifunctional Wound Healing Agent Calreticulin (CRT) in Combating Infection: Significance for Treating Diabetic Foot Ulcers. Wound Repair Regen. 2014, 22. A42.
(12) Pandya, U. M.; Manzanares, M. A.; Tellechea, A.; Egbuta, C.; Daubriac, J.; Jimenez-Jaramillo, C.; Samra, F.; Fredston-Hermann. A.; Saadipour, K.; Gold, L. I. Calreticulin Exploits TGF-β for Extracellular Matrix Induction Engineering a Tissue Regenerative Process. FASEB J. 2020, 34 (12), 15849-15874. doi.org/10.1096/fj.202001161R.
(13) Trengove, N. J.; Stacey, M. C.; Macauley. S.; Bennett. N.; Gibson, J.; Burslem, F.; Murphy. G.; Schultz, G. Analysis of the Acute and Chronic Wound Environments: The Role of Proteases and Their Inhibitors. Wound Repair Regen. 1999, 7 (6), 442-452. doi.org/10.1046/j.1524-475X.1999.00442.x.
(14) Lauer. G.; Sollberg. S.; Cole. M.; Flamme. I.; Stürzebecher. J.; Mann. K.; Krieg. T.; Eming. S. A. Expression and Proteolysis of Vascular Endothelial Growth Factor Is Increased in Chronic Wounds. J. Invest. Dermatol. 2000, 115 (1),12-18. doi.org/10.1046/j. 1523-1747.2000.00036.x.
(15) Schneider. L. A.; Korber. A.; Grabbe. S.; Dissemond. J. Influence of PH on Wound-Healing: A New Perspective for Wound-Therapy? Arch. Dermatol. Res. 2007, 298 (9), 413-420. doi.org/10.1007/s00403-006-0713-x.
(16) Gainza, G.; Villullas, S.; Pedraz, J. L.; Hernandez, R. M.; Igartua. M. Advances in Drug Delivery Systems (DDSs) to Release Growth Factors for Wound Healing and Skin Regeneration. Nanomedicine Nanotechnol. Biol. Med. 2015, 11 (6), 1551-1573. doi.org/10.1016/j.nano 2015.03.002.
(17) Kim, H. S.; Sun, X; Lee. J. H.; Kim, H. W.; Fu, X.; Leong. K. W. Advanced Drug Delivery Systems and Artificial Skin Grafts for Skin Wound Healing. Adv. Drug Deliv. Rev. 2019, 146, 209-239. doi.org/10.1016/j.addr.2018.12.014
(18) Saghazadeh. S.; Rinoldi, C.; Schot, M.; Kashaf, S. S.; Sharifi, F.; Jalilian. E.; Nuutila. K.; Giatsidis, G.; Mostafalu. P.; Derakhshandeh, H.; Yue, K.; Swieszkowski, W.; Memic. A.; Tamayol, A.; Khademhosseini, A. Drug Delivery Systems and Materials for Wound Healing Applications. Adv. Drug Deliv. Rev. 2018, 127, 138-166. doi.org/10.1016/j.addr.2018.04.008.
(19) Meinel, A. J.; Germershaus, O.; Luhmann, T.; Merkle. H. P.; Meinel, L. Electrospun Matrices for Localized Drug Delivery: Current Technologies and Selected Biomedical Applications. Eur. J. Pharm. Biopharm. 2012, 81 (1), 1-13. doi.org/10.1016/j.ejpb.2012.01.016.
(20) Sundaramurthi, D.; Krishnan, U. M.; Sethuraman. S. Electrospun Nanofibers as Scaffolds for Skin Tissue Engineering. Polym. Rev. 2014, 54 (2), 348-376. doi.org/10.1080/15583724.2014.881374.
(21) Abrigo. M.; McArthur, S. L.; Kingshott. P. Electrospun Nanofibers as Dressings for Chronic Wound Care: Advances. Challenges, and Future Prospects. Macromol. Biosci. 2014, 14 (6), 772-792. doi.org/10.1002/mabi.201300561.
(22) Yang, X.; Shah. J. D.; Wang. H. Nanofiber Enabled Layer-by-Layer Approach Toward Three-Dimensional Tissue Formation. Tissue Eng. Part A 2009, 15 (4), 945-956. doi.org/10.1089/ten.tea.2007.0280.
(23) Mahjour. S. B.; Fu, X.; Yang. X.; Fong. J.; Sefat, F.; Wang, H. Rapid Creation of Skin Substitutes from Human Skin Cells and Biomimetic Nanofibers for Acute Full-Thickness Wound Repair. Burns 2015, 41 (8), 1764-1774. doi.org/10.1016/j.burns.2015.06.011.
(24) Fu, X.; Xu. M.; Liu, J.; Qi, Y.; Li, S.; Wang, H. Regulation of Migratory Activity of Human Keratinocytes by Topography of Multiscale Collagen-Containing Nanofibrous Matrices. Biomaterials 2014, 35 (5), 1496-1506. doi org/10.1016/j.biomaterials.2013.11.013.
(25) Huang. C.; Fu, X.; Liu, J.; Qi, Y.; Li, S.; Wang, H. The Involvement of Integrin B1 Signaling in the Migration and Myofibroblastic Differentiation of Skin Fibroblasts on Anisotropic Collagen-Containing Nanofibers. Biomaterials 2012, 33 (6), 1791-1800. doi.org/10.1016/j.biomaterials.2011.11.025.
(26) Venugopal, J.; Ramakrishna, S. Biocompatible Nanofiber Matrices for the Engineering of a Dermal Substitute for Skin Regeneration. Tissue Eng. 2005, 11 (5-6), 847-854. doi.org/10.1089/ten.2005.11.847.
(27) Gautam, S.; Chou, C.-F.; Dinda, A. K.; Potdar, P. D.; Mishra, N. C. Surface Modification of Nanofibrous Polycaprolactone/Gelatin Composite Scaffold by Collagen Type I Grafting for Skin Tissue Engineering. Mater. Sci. Eng. C Mater. Biol. Appl. 2014, 34, 402-409. doi.org/10.1016/j.msec.2013.09.043.
(28) Yang, X.; Ogbolu, K. R.; Wang, H. Multifunctional Nanofibrous Scaffold for Tissue Engineering. J. Exp. Nanosci. 2008, 3 (4), 329-345. doi.org/10.1080/17458080701883707.
(29) Albright, V.; Xu. M.; Palanisamy, A.; Cheng, J.; Stack, M.; Zhang, B.; Jayaraman. A.; Sukhishvili, S. A.; Wang, H. Micelle-Coated, Hierarchically Structured Nanofibers with Dual-Release Capability for Accelerated Wound Healing and Infection Control. Adv. Healthc. Mater. 2018, 7 (11), e1800132. doi.org/10.1002/adhm.201800132.
(30) Cordelires, F. Manual Tracking.
(31) Thevenaz, P.; Ruttimann. U. E.; Unser, M. A Pyramid Approach to Subpixel Registration Based on Intensity. IEEE Trans. Image Process. 1998, 7 (1), 27-41. doi.org/10.1109/83.650848.
(32) Suarez-Arnedo, A.; Torres Figueroa. F.; Clavijo. C.; Arbelaez. P.; Cruz, J. C.; Muñoz-Camargo, C. An Image J Plugin for the High Throughput Image Analysis of in Vitro Scratch Wound Healing Assays. PLoS ONE 2020, 15 (7), e0232565. doi.org/10.1371/journal.pone.0232565.
(33) Stirling, D. R.; Swain-Bowden, M. J.; Lucas, A. M.; Carpenter, A. E.; Cimini. B. A.; Goodman, A. CellProfiler 4: Improvements in Speed, Utility and Usability. BMC Bioinformatics 2021, 22 (1), 433. doi.org/10.1186/s12859-021-04344-9.
(34) Pelton, R. W.; Saxena, B.; Jones, M.; Moses, H. L.; Gold. L. I. Immunohistochemical Localization of TGFβ1, TGFβ2, and TGFβ3 in the Mouse Embryo: Expression Patterns Suggest Multiple Roles during Embryonic Development. J. Cell Biol. 1991, 115 (4), 1091-1105. doi.org/10.1083/jcb.115.4.1091.
(35) Armbruster. D. A.; Pry, T. Limit of Blank, Limit of Detection and Limit of Quantitation. Clin. Biochem. Rev. 2008, 29 (Suppl 1), S49-S52.
(36) Bettinger, C. J.; Langer, R.; Borenstein, J. T. Engineering Substrate Topography at the Micro-and Nanoscale to Control Cell Function. Angew. Chem. Int. Ed. 2009, 48 (30), 5406-5415. doi.org/10.1002/anie.200805179.
(37) Jin, H.; Hong, Z.; Su. W.; Li, J. A Plant-Specific Calreticulin Is a Key Retention Factor for a Defective Brassinosteroid Receptor in the Endoplasmic Reticulum. Proc. Natl. Acad. Sci. 2009, 106 (32), 13612-13617. doi.org/10.1073/pnas.0906144106.
(38) Johnson, C. D. L.; D'Amato. A. R.; Gilbert, R. J. Electrospun Fibers for Drug Delivery after Spinal Cord Injury and the Effects of Drug Incorporation on Fiber Properties. Cells Tissues Organs 2016, 202 (1-2), 116-135. doi.org/10.1159/000446621.
(39) Lin, K.; Chua, K.-N.; Christopherson, G. T.; Lim. S.; Mao, H.-Q. Reducing Electrospun Nanofiber Diameter and Variability Using Cationic Amphiphilcs. Polymer 2007, 48 (21), 6384-6394. doi.org/10.1016/j.polymer.2007.08.056.
(40) Fon, D.; Zhou, K.; Ercole. F.; Fehr, F.; Marchesan, S.; Minter, M. R.; Crack, P. J.; Finkelstein, D. I.; Forsythe. J. S. Nanofibrous Scaffolds Releasing a Small Molecule BDNF-Mimetic for the Re-Direction of Endogenous Neuroblast Migration in the Brain. Biomaterials 2014, 35 (9), 2692-2712. doi.org/10.1016/j.biomaterials.2013.12.016.
(41) Woodard, L. N.; Grunlan, M. A. Hydrolytic Degradation and Erosion of Polyester Biomaterials ACS Macro Lett. 2018, 7 (8), 976-982. doi.org/10.1021/acsmacrolett 8b00424.
(42) Chou, S.-F.; Carson. D.; Woodrow, K. A. Current Strategies for Sustaining Drug Release from Electrospun Nanofibers. J. Controlled Release 2015, 220 (Pt B), 584-591. doi.org/10.1016/j.jconrel.2015.09.008.
(43) Isabela Avila-Rodriguez, M.; Melendez-Martinez, D.; Licona-Cassani, C.; Manuel Aguilar-Yañez, J.; Benavides. J.; Lorena Sanchez, M. Practical Context of Enzymatic Treatment for Wound Healing: A Secreted Protease Approach (Review). Biomed. Rep. 2020, 13 (1), 3-14. doi.org/10.3892/br.2020.1300.
(44) McCarty, S. M.; Percival, S. L. Proteases and Delayed Wound Healing. Adv. Wound Care 2013, 2 (8), 438-447. doi.org/10.1089/wound.2012.0370.
(45) Singer, A. J.; Clark. R. A. F. Cutaneous Wound Healing. N. Engl. J. Med. 1999, 341 (10), 738-746. doi.org/10.1056/NEJM199909023411006.
(46) Stutchbury, B.; Atherton. P.; Tsang. R.; Wang, D.-Y.; Ballestrem, C. Distinct Focal Adhesion Protein Modules Control Different Aspects of Mechanotransduction. J. Cell Sci. 2017, 130 (9), 1612-1624. doi.org/10.1242/jcs.195362.
(47) Bays, J. L.; DeMali, K. A. Vinculin in Cell-Cell and Cell-Matrix Adhesions. Cell. Mol. Life Sci. 2017, 74 (16), 2999-3009. doi.org/10.1007/s00018-017-2511-3.
(48) Kim. D.-H.; Wirtz, D. Focal Adhesion Size Uniquely Predicts Cell Migration. FASEB J. 2013, 27 (4), 1351-1361. doi.org/10.1096/fj.12-220160.
(49) Ridley, A. J. Life at the Leading Edge. Cell 2011, 145 (7), 1012-1022. doi.org/10.1016/j.cell.2011.06.010.
(50) Wozniak, M. A.; Modzelewska. K.; Kwong. L.; Kecly, P. J. Focal Adhesion Regulation of Cell Behavior. Biochim. Biophys. Acta 2004, 1692 (2), 103-119. doi.org/10.1016/j.bbamer.2004.04.007.
(51) Zhang. Y.; Guo, W.; Bi, M.; Liu. W.; Zhou, L.; Liu, H.; Yan. F.; Guan, L.; Zhang. J.; Xu. J. Migrasomes: From Biogenesis, Release, Uptake, Rupture to Homeostasis and Diseases. Oxid. Med. Cell. Longev. 2022, 2022, 4525778. doi.org/10.1155/2022/4525778.
(52) Gold, L. I.; Eggleton, P.; Sweetwyne, M. T.; Van Duyn, L. B.; Greives, M. R.; Naylor. S.-M.; Michalak, M.; Murphy-Ulrich, J. E. Calreticulin: Non-endoplasmic Reticulum Functions in Physiology and Disease. FASEB J. 2009, 24 (3), 665-683. doi.org/10.1096/fj.09-145482.
(53) Huttenlocher, A.; Horwitz, A. R. Integrins in Cell Migration. Cold Spring Harb Perspect Biol. 2011, 3 (9), a005074. doi.org/10.1101/cshperspect.a005074.
(54) Repesh, L. A.; Fitzgerald, T. J.; Furcht, L. T. Fibronectin Involvement in Granulation Tissue and Wound Healing in Rabbits. J. Histochem. Cytochem. 1982, 30 (4), 351-358. doi.org/10.1177/30.4.6174568.
(55) Pace, R. T.; Burg, K. J. L. Toxic Effects of Resazurin on Cell Cultures. Cytotechnology 2015, 67 (1), 13-17. doi.org/10.1007/s10616-013-9664-1.
(56) Zhang, K.; Kramer, R. H. Laminin 5 Deposition Promotes Keratinocyte Motility. Exp. Cell Res. 1996, 227 (2), 309-322. doi.org/10.1006/exer.1996.0280.
(57) Roshan, A.; Murai, K.; Fowler, J.; Simons, B. D.; Nikolaidou-Neokosmidou, V.; Jones. P. H. Human Keratinocytes Have Two Interconvertible Modes of Proliferation. Nat. Cell Biol. 2016, 18 (2), 145-156. doi.org/10.1038/ncb3282.
(58) Park, S. A.; Raghunathan, V. K.; Shah, N. M.; Teixeira, L.; Motta, M. J.; Covert. J.; Dubielzig, R.; Schurr, M.; Isseroff, R. R.; Abbott. N. L.; McAnulty, J.; Murphy, C. J. PDGF-BB Does Not Accelerate Healing in Diabetic Mice with Splinted Skin Wounds. PLOS ONE 2014, 9 (8), e104447. doi.org/10.1371/journal.pone.0104447.
(59) Papanas, D.; Maltezos, E. Benefit-Risk Assessment of Becaplermin in the Treatment of Diabetic Foot Ulcers. Drug Saf. 2010, 33 (6), 455-461. doi.org/10.2165/11534570-000000000-00000.
(60) Stack, M. E. Multifunctional, Biomimetic, Acellular Skin Grafts for the Treatment of Chronic, Diabetic Foot Ulcers. Doctoral Dissertation, Stevens Institute of Technology, Hoboken, N J, 2022.
(61) Pandya, U. M.; Egbuta. C.; Abdullah Norman, T. M.; Chiang, C.-Y. (Edward); Wiersma, V. R.; Panchal, R. G.; Bremer, F.; Eggleton, P.; Gold, L. I. The Biophysical Interaction of the Danger-Associated Molecular Pattern (DAMP) Calreticulin with the Pattern-Associated Molecular Pattern (PAMP) Lipopolysaccharide. Int. J. Mol. Sci. 2019, 20 (2), 408. doi.org/10.3390/ijms20020408.
(62) Fu X, Wang H. Spatial arrangement of polycaprolactone/collagen nanofiber scaffolds regulates the wound healing related behaviors of human adipose stromal cells. Tissue engineering Part A. 2012; 18(5-6):631-42.
(63) Pandya UM, Gold LI. The Novel Biotherapeutic Calreticulin (CRT) Corrects Multiple Defects of Non-Healing Diabetic Wounds. J Derm Clin Res. 2016; 4(5):1083-93.
(64) Zimmerman K A, Graham L V, Pallero M A, Murphy-Ullrich J E. Calreticulin regulates transforming growth factor-beta-stimulated extracellular matrix production. J Biol Chem. 2013;288 (20):14584-98.
(65) Vass P. Szabó E, Domokos A, Hirsch E, Galata D, Farkas B, et al. Scale-up of electrospinning technology: Applications in the pharmaceutical industry. Wiley interdisciplinary reviews Nanomedicine and nanobiotechnology. 2020;12(4):c1611.
(66) Pallero M A, Elzie C A, Chen J, Mosher D F, Murphy-Ullrich J E. Thrombospondin 1 binding to calreticulin-LRP1 signals resistance to anoikis. Faseb J. 2008.
(67) Woodley D T, Fan J, Cheng C F, Li Y, Chen M, Bu G, et al. Participation of the lipoprotein receptor LRP1 in hypoxia-HSP90alpha autocrine signaling to promote keratinocyte migration. J Cell Sci. 2009;122(Pt 10):1495-8.
(68) Wang L, Song Y. Molecular insights into the effect of an apoptotic raft-like bilayer on the conformation and dynamics of calreticulin. Biochimica et biophysica acta Biomembranes. 2020;1862 (2):183146.
(69) Gold L, Williams D, Groenendyk J, Michalak M, Eggleton P. Unfolding the complexities of ER chaperones in health and disease: report on the 11th international calreticulin workshop. Cell Stress Chaperones. 2015;20(6):875-83.
(70) Gardai S J, McPhillips K A, Frasch S C, Janssen W J, Starefeldt A, Murphy-Ullrich J E, et al. Cell-surface calreticulin initiates clearance of viable or apoptotic cells through trans-activation of LRP on the phagocyte. Cell. 2005;123(2):321-34.
(71) Michalak M, Groenendyk J, Szabo E, Gold L I, Opas M. Calreticulin, a multi-process calcium-buffering chaperone of the endoplasmic reticulum. Biochem J. 2009;417(3):651-66.
(72) Michalak M, Robert Parker J M, Opas M, Ca2+ signaling and calcium binding chaperones of the endoplasmic reticulum. Cell Calcium. 2002;32(5-6):269-78.
(73) He M C, Wang J, Wu J, Gong F Y, Hong C, Xia Y, et al. Immunological activity difference between native calreticulin monomers and oligomers. PLoS One. 2014;9(8):e105502.
(74) Huang S H, Zhao L X, Hong C, Duo C C, Guo B N, Zhang L J, et al. Self-oligomerization is essential for enhanced immunological activities of soluble recombinant calreticulin. PLOS One. 2013;8(6):e64951.
(75) Hong C, Zhang T, Gao X M. Recombinant murine calreticulin fragment 39-272 expands CD1d(hi)CD5+ IL-10-secreting B cells that modulate experimental autoimmune encephalomyelitis in C57BL/6 mice. Mol Immunol. 2013;55(3-4):237-46.
(76) Theocharidis, G. et al., 2022. Nature Communications, 13:181, 27801-8.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A polymeric matrix comprising calreticulin, or a functional fragment or derivative thereof.

2. The polymeric matrix of claim 1, wherein the derivative of calreticulin is a recombinant protein comprising calreticulin or a functional fragment of calreticulin.

3. The polymeric matrix of claim 1 or 2, wherein the functional fragment is N-, P- or C-domain of calreticulin.

4. The polymeric matrix of any one of claims 1-3, wherein the polymeric matrix comprises a synthetic polymer, natural polymer, or a combination thereof.

5. The polymeric matrix of claim 4, wherein the synthetic polymer comprises polycaprolactone (PCL), polylactic acid (PLA), poly(lactic-co-glycolic acid) (PLGA), poly(l-lactic acid)-co-poly(ε-caprolactone) (PLCL), polyphosphazene, poly-N-vinylpyrrolidone, polyglycolic acid, polydimethylsiloxane, poly(ethylene oxide)-poly(butylene terephthalate), nylon, polyvinyl alcohol (PVA), polyethylene glycol (PEG), or a combination thereof.

6. The polymeric matrix of claim 4, wherein the natural polymer comprises collagen, chitosan, gelatin, hyaluronic acid, chondroitin sulfate, silk fibroin, elastin, tropoelastin, fibrin, fibrinogen, carboxymethyl cellulose, cellulose, decellularized tissue matrix, or a combination thereof.

7. The polymeric matrix of claim 4, wherein the polymeric matrix comprises PCL and collagen.

8. The polymeric matrix of claim 6 or 7, wherein the collagen comprises type I collagen (Col1), type II collagen (Col2), type III collagen (Col3), type IV collagen (Col4), type V collagen (Col5), type VII collagen (Col7), or a combination thereof.

9. The polymeric matrix of any one of claims 6-8, wherein the collagen comprises type I collagen (Col1).

10. The polymeric matrix of any one of claims 7-10, wherein the polymeric matrix comprises PCL and collagen at a weight-to-weight ratio of about 1:10 to 10:1.

11. The polymeric matrix of claim 10, wherein the polymeric matrix comprises PCL and collagen at a weight-to-weight ratio of about 3:1.

12. The polymeric matrix of any one of claims 1-11, wherein the polymeric matrix is in the form of nanofibers, foams, sponges, nonwoven meshes, spheres, hydrogels, or 3D printed filament structures.

13. The polymeric matrix of claim 12, wherein the polymeric matrix is in the form of nanofibers.

14. The polymeric matrix of claim 12, wherein the nanofibers have a diameter of about 10-1000 nm

15. The polymeric matrix of claim 13, wherein the nanofibers have a diameter of about 100-500 nm

16. The polymeric matrix of claim 14, wherein the nanofibers have a diameter of about 330 nm.

17. The polymeric matrix of any one of claims 1-16, wherein the polymeric matrix comprises a concentration of calreticulin at about 1 pg-100 mg/mL.

18. The polymeric matrix of claim 17, wherein the polymeric matrix comprises a concentration of calreticulin at about 1pg-10 mg/mL.

19. The polymeric matrix of claim 17 or 18, wherein the polymeric matrix comprises a concentration of calreticulin at about 100 ng/ml.

20. The polymeric matrix of any one of claims 1-19, wherein the polymeric matrix further comprises an additional agent.

21. The polymeric matrix of claim 20, wherein the polymeric matrix further comprises a cytokine, a growth factor, a glycosaminoglycan, a heat shock protein, a proteoglycan, a glycoprotein, syndecan, gelatin, or any mixtures thereof.

22. The polymeric matrix of claim 21, wherein said glycosaminoglycan is hyaluronic acid.

23. The polymeric matrix of claim 21, wherein said proteoglycan is perlecan or heparin sulfate.

24. The polymeric matrix of claim 21, wherein said glycoprotein is fibronectin.

25. The polymeric matrix of claim 21, wherein said growth factor is selected from the group consisting of a platelet-derived growth factor, vascular endothelial growth factor, fibroblast growth factor, epidermal growth factor, transforming growth factor-beta, and any mixtures thereof.

26. The polymeric matrix of any one of claims 1-25, wherein the polymeric matrix has a three-dimensional structure.

27. The polymeric matrix of any one of claims 1-26, wherein the polymeric matrix is produced by electrospinning.

28. The polymeric matrix of any one of claims 1-27, wherein the polymeric matrix has one or more of the following characteristics:
1) induces proliferation and/or migration of keratinocytes;
2) induces proliferation and/or migration of fibroblasts;
3) induces expression of CD68 in monocytes;
4) induces TGF-β1, fibronectin, collagen, laminin-5, p-FAK, integrin α5, and/or integrin β1 protein levels in fibroblasts;
5) promotes a polarized cell shape, which is the morphlogial phenotype of a motogenic cell replete with lamellipodia and filopodia;
6) induces elongated, oriented, and aligned cells on the CRT-NFs where CRT is presented at the basal side of the cell as well as the apical side;
7) induces dermal fibroblasts from the plantar foot of a non-healing wounds to adopt the phenotype of dermal fibroblasts from the plantar foot of a healing wound; and
8) induces tissue regeneration upon application to a mammal acute or chronic wound.

29. A method of producing a polymeric matrix, comprising:
c) mixing calreticulin, or a functional fragment or derivative thereof, within a polymeric solution; and
d) fabricating the polymeric matrix from the solution generated in step (a) using electrospinning.

30. The method of claim 29, wherein the derivative of calreticulin is a recombinant protein comprising calreticulin or a functional fragment of calreticulin.

31. The method of claim 29 or 30, wherein the functional fragment is N-, P- or C-domain of calreticulin.

32. The method of any one of claims 29-31, wherein the calreticulin, or functional fragment or derivative thereof, is present in a solution comprising calreticulin, or functional fragment or derivative thereof, and a buffer.

33. The method of claim 32, wherein the buffer comprises an organic amine and a metal halide salt at a pH from about 6 to about 8.

34. The method of claim 33, wherein the organic amine is tromethamine.

35. The method of claim 33, wherein the metal halide salt is CaCl₂.

36. The method of claim 32, wherein the buffer comprises saline, or PBS (phosphate buffered saline).

37. The method of any one of claims 29-36, wherein the polymeric solution comprises a synthetic polymer, natural polymer, or a combination thereof.

38. The method of claim 37, wherein the synthetic polymer comprises polycaprolactone (PCL), polylactic acid (PLA), poly(lactic-co-glycolic acid) (PLGA), poly(l-lactic acid)-co-poly(ε-caprolactone) (PLCL), polyphosphazene, poly-N-vinylpyrrolidone, polyglycolic acid, polydimethylsiloxane, poly(ethylene oxide)-poly(butylene terephthalate), nylon, polyvinyl alcohol (PVA), polyethylene glycol (PEG), or a combination thereof.

39. The method of claim 37, wherein the natural polymer comprises collagen, chitosan, gelatin, hyaluronic acid, chondroitin sulfate, silk fibroin, elastin, tropoelastin, fibrin, fibrinogen, carboxymethyl cellulose, cellulose, decellularized tissue matrix, or a combination thereof.

40. The method of claim 37, wherein the polymeric solution comprises PCL and collagen.

41. The method of claim 40, wherein the collagen comprises type I collagen (Col1), type II collagen (Col2), type III collagen (Col3), type IV collagen (Col4), type V collagen (Col5), or a combination thereof.

42. The method of any one of claims 39-41, wherein the collagen comprises type I collagen (Col1).

43. The method of any one of claims 39-42, wherein the polymeric matrix comprises PCL and collagen at a weight-to-weight ratio of about 1:10 to 10:1.

44. The method of claim 43, wherein the polymeric solution comprises PCL and collagen at a weight-to-weight ratio of about 3:1.

45. The method of any one of claims 29-44, wherein the polymeric solution comprises a solvent.

46. The method of claim 45, wherein the solvent is 1,1,1,3,3,3-hexafluoro-2-propanol (HFIP), trifluoroacetic acid, dichloromethane, or chloroform.

47. The method of any one of claims 29-46, wherein the mixing step (a) is carried out at a temperature of about 0 to about 25° C.

48. The method of any one of claims 29-47, wherein the mixing step (a) is carried out at a temperature of about 4 to about 10° C.

49. The method of any one of claims 29-47, wherein the mixing step (a) is carried out at a temperature of about 4° C.

50. The method of any one of claims 29-49, wherein the final solution generated in step (a) comprises PCL and the solvent at a weight-to-volume ratio of about 1-20%.

51. The method of any one of claims 29-50, wherein the final solution generated in step (a) comprises PCL and the solvent at a weight-to-volume ratio of about 10%.

52. The method of any one of claims 29-51, wherein the fabrication step (b) is carried out at a temperature of about 20° C. to about 30° C.

53. The method of any one of claims 29-52, wherein the fabrication step (b) is carried out at a relative humidity of about 10% to about 60%.

54. The method of any one of claims 29-53, wherein the electrospinning in step (b) is carried out using an electric field of about 0.5-20 kV/cm.

55. The method of any one of claims 29-54, wherein the electrospinning in step (b) is carried out using an electric field of about 1 kV/cm.

56. The method of any one of claims 29-55, wherein the polymeric matrix is collected at a distance of 2 mm-50 cm.

57. The method of any one of claims 29-56, wherein step (a) further comprises mixing an additional agent within the polymeric solution.

58. The method of claim 57, wherein the additional agent is a cytokine, a growth factor, a glycosaminoglycan, a heat shock protein, a proteoglycan, a glycoprotein, syndecan, gelatin or any mixtures thereof.

59. The method of claim 57, wherein said glycosaminoglycan is hyaluronic acid.

60. The method of claim 57, wherein said proteoglycan is perlecan or heparin sulfate.

61. The method of claim 21, wherein said glycoprotein is fibronectin.

62. The method of claim 57, wherein said growth factor is selected from the group consisting of a platelet-derived growth factor, vascular endothelial growth factor, fibroblast growth factor, epidermal growth factor, transforming growth factor-beta, and any mixtures thereof.

63. A polymeric matrix produced by the method of any one of claims 29-62.

64. A method for treating a wound in a subject in need thereof, said method comprises applying the polymeric matrix of any one of claims 1-28 and 63 to the wound in the subject.

65. The method of claim 64, wherein the polymeric matrix is applied topically or internally to the wound.

66. The method of claim 64 or 65, wherein the wound is an acute wound or a chronic wound.

67. The method of claim 66, wherein the acute wound is a burn, injury, or surgical intervention.

68. The method of claim 66, wherein the chronic wound is a chronic diabetic wound, a venous or arterial stasis ulcer, a pressure ulcer, or an ulcer resulting from sickle cell disease (SCU).

69. The method of claim 68, wherein the chronic diabetic wound is diabetic foot ulcer (DFU).

70. The method of any one of claims 64-69, wherein the wound is a post-surgical wound or an internal wound.

71. A method for promoting healing of a chronic diabetic wound in a subject in need thereof, said method comprises applying the polymeric matrix of any one of claims 1-28 and 63 to the wound in the subject.

72. The method of claim 71, wherein applying the polymeric matrix to the wound results in tissue regeneration in which neogenic epidermal appendage appear and scarring is reduced or eliminated.

73. A method of inducing migration and/or proliferation of diabetic fibroblasts in a chronic diabetic wound in a subject in need thereof, said method comprises contacting the fibroblasts with the polymeric matrix of any one of claims 1-28 and 63.

74. The method of any one of claims 71-73, wherein the diabetic wound is diabetic foot ulcer (DFU).

75. The method of any one of claims 71-74, wherein the polymeric matrix is applied topically or internally to the wound.

76. The method of any one of claims 64-75, further comprising administering a cytokine, a chemokine, a growth factor, a glycosaminoglycan, a heat shock protein, a proteoglycan, a glycoprotein, gelatin, syndecan or any mixtures thereof.

77. The method of claim 76, wherein said glycosaminoglycan is hyaluronic acid.

78. The method of claim 76, wherein said proteoglycan is perlecan or heparin sulfate.

79. The method of claim 76, wherein said glycoprotein is fibronectin.

80. The method of claim 76, wherein said growth factor is selected from the group consisting of a platelet-derived growth factor, vascular endothelial growth factor, fibroblast growth factor, epidermal growth factor, transforming growth factor-beta, and any mixtures thereof.

81. The method of any one of claims 64-80, wherein the subject is mammal.

82. The method of any one of claims 64-81, wherein the subject is human.
